(19) United States
(12) Patent Application Publication
(10) Pub. No.: US 2025/0232884 A1
(43) Pub. Date: Jul. 17, 2025

(54) MACHINE LEARNING-BASED PREDICTIVE ANALYTICS FOR REFERRAL DIAGNOSES

(71) Applicant: MatrixCare, Inc., Bloomington, MN (US)

(72) Inventor: Denver Michael WEST, Republic, MO (US)

(21) Appl. No.: 19/020,616

(22) Filed: Jan. 14, 2025

Related U.S. Application Data
(60) Provisional application No. 63/621,702, filed on Jan. 17, 2024.

(51) Int. Cl. G16H 50/70; G16H 40/20

(52) U.S. Cl. CPC G16H 50/70; G16H 40/20

(57) ABSTRACT

Techniques for machine learning-based data evaluation are provided. A patient referral of a patient to a healthcare service is accessed, and a referral condition of the patient is determined based on the patient referral. Using a machine learning model, a prediction indicating one or more referral outcomes is generated based on the first referral condition. Acceptance of the patient referral to the healthcare service is facilitated based on the prediction.

### CROSS-REFERENCE TO RELATED APPLICATIONS

This application claims priority to U.S. Provisional Patent Application No. 63/621,702, filed Jan. 17, 2024, the entire content of which is incorporated herein by reference in its entirety.

### INTRODUCTION

Aspects of the present disclosure relate to machine learning. More specifically, aspects of the present disclosure relate to training and using machine learning to guide healthcare service referral and treatment.

In many locales, a wide variety of healthcare settings or services exist, often providing services ranging from general healthcare to highly specialized care. For example, general practitioners (also referred to as primary care physicians) generally correspond to healthcare providers that focus on patients' overall health, and are often prepared to provide treatment for acute care (e.g., for illness and injury) as well as providing preventative care. Specialists generally correspond to providers that provide more focused or specialized care, such as specializing in dermatological care, oncology, and the like. In some cases, more general healthcare providers (e.g., primary care doctors) provide specialist referrals (e.g., instructing a patient to seek further care from a specialist) when such specialists are better-suited to diagnose and/or treat a given concern. Similarly, specialists may refer patients to other specialists, providers of any specificity may refer patients to other services (e.g., home health services), and the like.

However, in many locales, there are a large number of healthcare providers that provide similar or overlapping services. When a provider desires to refer a patient to another healthcare provider or service, there are a tremendous number of factors that may weigh on the decision, such as the proximity of the referral target (e.g., how close the patient lives to the target), whether the referring target is accepting patients, and the like. Unfortunately, conventional approaches to referrals generally rely on the initial care provider selecting the referral target based largely on their own subjective preferences (e.g., preferring one specialist over another) without consideration of how well suited the target is for the specific patient. This results in sub-optimal care. Further, when a referral is received, the target provider must decide whether to accept the patient. Such determinations are often similarly subjective and/or are made based on the availability of the practice to accept a new patient, without consideration for the particular match between the specific referred patient and the service.

Improved systems and techniques to make and evaluate referrals are needed.

### SUMMARY

According to one embodiment presented in this disclosure, a method is provided. The method includes: accessing a first patient referral of a first patient to a first healthcare service; determining, based on the first patient referral, a first referral condition of the first patient; generating, using a first machine learning model, a first prediction indicating one or more referral outcomes based on the first referral condition; and facilitating acceptance of the first patient referral to the first healthcare service based on the first prediction.

According to a second embodiment of the present disclosure, a method is provided. The method includes: accessing a first set of patient referrals to a first healthcare service, each respective patient referral indicating a respective referral condition; determining first outcome data comprising, for each respective patient referral of the first set of patient referrals, one or more respective referral outcomes of a corresponding patient after transitioning to the first healthcare service; training a first machine learning model to predict one or more referral outcomes based on the first set of patient referrals and the first outcome data; and deploying the first machine learning model to process new patient referrals to the first healthcare service.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below. The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for using machine learning to evaluate patient referrals, according to one embodiment of the present disclosure.

FIG. 2 depicts an example workflow for generating predicted outcomes for patient referrals, according to one embodiment of the present disclosure.

FIG. 3 depicts an example workflow for generating predicted outcomes and revised referrals for patient referrals, according to one embodiment of the present disclosure.

FIG. 4 depicts an example workflow for generating predicted outcomes for patient referrals and refining machine learning models based on subsequent outcome feedback, according to one embodiment of the present disclosure.

FIG. 5 depicts an example workflow for training machine learning models to evaluate patient referrals, according to one embodiment of the present disclosure.

FIG. 6 is a flow diagram depicting an example method for evaluating referrals using machine learning, according to one embodiment of the present disclosure.

FIG. 7 is a flow diagram depicting an example method for determining relevant patient data for evaluating referrals, according to one embodiment of the present disclosure.

FIG. 8 is a flow diagram depicting an example method for generating predicted outcomes using service-specific machine learning models, according to one embodiment of the present disclosure.

FIG. 9 is a flow diagram depicting an example method for generating predicted outcomes using service-agnostic machine learning models, according to one embodiment of the present disclosure.

FIG. 10 is a flow diagram depicting an example method for generating proposed healthcare modifications to improve referral outcomes, according to one embodiment of the present disclosure.

FIG. 11 is a flow diagram depicting an example method for generating predicted outcomes using machine learning, according to one embodiment of the present disclosure.

FIG. 12 is a flow diagram depicting an example method for updating machine learning models based on referral outcomes, according to one embodiment of the present disclosure.

FIG. 13 is a flow diagram depicting an example method for training machine learning models to predict referral outcomes, according to one embodiment of the present disclosure.

FIG. 14 is a flow diagram depicting an example method for using machine learning models to evaluate referral conditions, according to one embodiment of the present disclosure.

FIG. 15 is a flow diagram depicting an example method for training machine learning models to evaluate referral conditions, according to one embodiment of the present disclosure.

FIG. 16 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved referral evaluation and outcome prediction using machine learning.

In some embodiments, when a patient referral is received, relevant patient data can be accessed and evaluated using one or more machine learning models to predict how well-suited the referral target is with respect to the patient at the particular point in time. For example, when a first healthcare service or provider (a referring entity) refers a patient to a second healthcare service or provider (the referral target), a computing system associated with the referral target may evaluate the referral and any other relevant data to predict outcome(s) that will occur if the referral target accepts the referral.

Generally, the particular outcomes that are predicted may vary depending on the particular implementation, and each prediction generally relates or corresponds to how well-suited the referral target is to accept the referral. By way of example and not limitation, the predicted outcomes may include a prediction as to whether the patient will recover from the condition for which the patient was referred to the referral target (referred to as a referral condition) (e.g., the probability of recovery, a binary recovery prediction, and the like), a predicted recovery timeline (e.g., indicating a predicted length of time until the patient recovers), a prediction as to whether the patient will be hospitalized during treatment by the referral target, a prediction as to whether the first patient will become septic during treatment by the referral target, and the like.

Such outcome predictions may be valuable in determining whether the referral target is suitable for the patient. For example, if the patient is predicted to become septic if accepted by the referral target, but the probability of sepsis would be lower if the patient entered a different healthcare service, the referral target may not be a good fit. In some aspects, patient recovery may be a useful metric for conditions where recovery is anticipated or desired. However, in some aspects, recovery may not be a valid outcome (e.g., for referral to hospice care). In some embodiments, therefore, the system may similarly predict other outcomes such as whether the patient would be comfortable at the referral target. Generally, the particular predictions may vary depending on the particular implementation.

In some embodiments, based on the machine learning-based predictions, the computing system may determine whether to accept or decline the referral (or may otherwise facilitate or assist in this determination). For example, based on the predictions and one or more defined acceptance rules, the system may determine that the referral should likely be declined (e.g., because the predicted probability of recovery is below a threshold criteria). In some embodiments, the system may indicate or output this recommendation to a user (e.g., a healthcare provider associated with the referral target), allowing the user to determine whether to accept or decline.

In some embodiments, in addition to or instead of suggesting whether the referral should be accepted, the system may output the predictions themselves, allowing the user to accept or decline the referral. In some embodiments, in addition to or instead of outputting the predictions and/or suggestion, the system may perform further analysis to determine what changes (if any) would make the referral target a better fit for the patient (or for similarly situated patients). For example, in response to predicting that another referral target would result in improved outcomes, the system may compare the two services to identify difference(s) that may cause this difference in prediction, and generate appropriate suggestions (e.g., indicating that hiring one or more additional registered nurses (RNs) may improve outcomes for similarly situated patients).

In some embodiments, in addition to or instead of outputting the predictions and/or suggestions, the system may indicate which other healthcare service(s) would be better-suited for the referral patient. In this way, the user can quickly update or generate a new referral to the indicated service(s), improving patient outcomes.

In some embodiments, rather than the referral target evaluating the referral, the referring entity may evaluate multiple alternative services to determine where the patient should be referred to. For example, the referring entity (e.g., a general practitioner) may identify relevant target services (e.g., all oncology specialists in a defined area proximate to the patient), and evaluate each alternative using machine learning to predict which service(s) should be used as the referral target for the specific patient.

Generally, using machine learning, embodiments of the present disclosure can provide a wide variety of improvements to the computational systems, as well as to other systems and fields. For example, the patient outcomes may be substantially improved, such as through increased probability of recovery, reduced probability of further health complications, and the like. In this way, embodiments of the present disclosure improve the healthcare field. As another example, resource waste may be substantially reduced, such as by ensuring that patients are referred to (more) optimal service providers, preventing less optimal providers from wasting resources that could be better spent on other patients who are better suited for the particular provider. In this way, embodiments of the present disclosure similarly improve the process of allocating and providing healthcare services.

As yet another example, the operations of the computing systems themselves may be improved, such as by using more efficient data retrieval and evaluations, as compared to conventional solutions. For example, some conventional systems require users to manually locate and retrieve any relevant data to evaluate referrals, which can impose substantial computational burden on the systems, such as through increased bandwidth from repeated queries and data exchanges between storage repositories, increased power consumption caused by such manual searching, increased heat generation and power consumption caused by outputting such data visually to the user (e.g., requiring that a monitor be powered on and outputting data for a long period of time so the user can manually review the data, which consumes substantial energy and generates substantial heat), and the like. In contrast, embodiments of the present disclosure can use targeted and specific data retrieval processes (reducing bandwidth and duplicative exchanges). Further, by using machine learning to generate predictions, the resources needed to facilitate the decision process are reduced substantially (e.g., display devices need not consume power until the evaluations are complete, and the user need only review the output briefly to make an informed decision). Embodiments of the present disclosure thereby provide substantial improvements to the operations of the computer itself.

### Example Environment for Using Machine Learning to Evaluate Patient Referrals

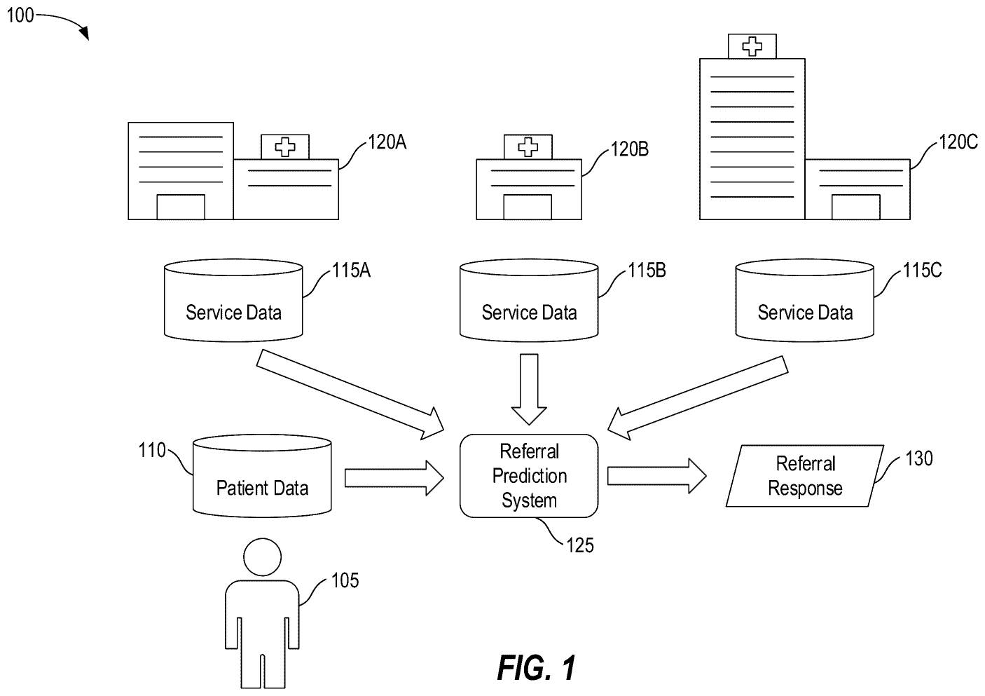

FIG. 1 depicts an example environment 100 for using machine learning to evaluate patient referrals, according to one embodiment of the present disclosure.

In the illustrated example, a referral prediction system 125 accesses a variety of data, including patient data 110 and service data 115 from a variety of service providers 120, to generate referral responses 130. As used herein, “accessing” data generally corresponds to receiving, requesting, retrieving, generating, collecting, obtaining, or otherwise gaining access to the data. For example, the referral prediction system 125 may access data from local storage or memory, from one or more repositories or other systems located remotely from the referral prediction system 125 (e.g., via one or more networks, such as the Internet), and the like.

In the illustrated example, the referral prediction system 125 uses service data 115 specific to each service provider 120 to drive referral responses 130. Specifically, the service provider 120A has associated service data 115A, the service provider 120B has associated service data 115B, and the service provider 120C has associated service data 115C. By using service-specific data, the referral prediction system 125 may generate service-specific predictions (e.g., predicting outcomes with respect to each specific service provider 120), allowing for improved referral responses 130. Although three service providers 120 are depicted for conceptual clarity, in embodiments, the referral prediction system 125 may evaluate data for any number and variety of service providers.

In some embodiments, the referral prediction system 125 trains one or more machine learning models based on the service data 115. For example, in some embodiments, the referral prediction system 125 trains a respective machine learning model for each respective service provider 120. That is, the referral prediction system 125 may train a first machine learning model for the service provider 120A based on the corresponding service data 115A, a second machine learning model for the service provider 120B based on the corresponding service data 115B, and a third machine learning model for the service provider 120C based on the corresponding service data 115C. To generate a service-specific prediction, the referral prediction system 125 may evaluate the patient data 110 using the specific model(s) trained for the specific service provider 120.

In some embodiments, the referral prediction system 125 may train one or more machine learning models to be used across multiple service providers 120. For example, the referral prediction system 125 may train the model(s) to receive, as part of its input, an indication of the particular service provider 120 for which the predictions should be generated.

In some embodiments, the referral prediction system 125 trains outcome-specific models. For example, for each respective outcome of interest, the referral prediction system 125 may train one or more machine learning models to specifically predict the respective outcome. In some embodiments, the referral prediction system 125 may train a model to predict multiple outcomes.

In the illustrated example, once the machine learning model(s) are trained (as discussed in more detail below), the referral prediction system 125 may process patient data 110 (e.g., corresponding to a patient 105) using the model(s) to generate the referral response 130. For example, when the patient 105 is referred to a healthcare provider (e.g., one or more of the service providers 120), the referral prediction system 125 may evaluate corresponding patient data 110 using the machine learning model(s) to generate the referral response.

The patient data 110 generally includes any relevant information that is used to evaluate referral targets with respect to the patient 105. For example, in some embodiments, the patient data 110 may include the referral condition(s) (e.g., the medical condition(s) or concern(s) for which the patient 105 is being referred to the target), comorbidities of the patient 105 (e.g., other diagnoses or conditions the patient has), demographics of the patient 105 (e.g., their age, weight, and the like), medications the patient 105 consumes, allergies the patient 105 has, the region or locale where the patient 105 lives and/or where the services will be provided, and the like.

In some embodiments, some or all of the patient data 110 is included in or associated with the referral itself. In some embodiments, the referral may include less data (e.g., indicating only the identity of the patient 105 and the referral condition(s), and the referral prediction system 125 may retrieve any other relevant data (e.g., from one or more healthcare data repositories) based on the patient identifier. In some embodiments, the referral may indicate a specific target service provider 120, and the referral prediction system 125 may generate outcome predictions for the target provider based on the patient data 110. In some embodiments, the referral prediction system 125 may additionally evaluate alternative service providers 120 for the patient 105. In some embodiments, the referral does not specify a particular provider, and instead indicates the type(s) of care for which the patient is being referred. For example, the referral may indicate that the patient 105 is being referred for dermatological services. In response, the referral prediction system 125 may identify alternative service providers 120 providing such services, and evaluate each using the machine learning models to generate predictions and identify the best fit.

In an embodiment, the referral response 130 can generally include a wide variety of information in response to the referral. In some embodiments, the referral response 130 includes the one or more prediction(s) generated for one or more service providers 120. For example, the referral response 130 may indicate the predicted probabilities of recovery if the patient 105 is admitted to one or more of the service providers 120. In some embodiments, the referral response 130 indicates the best or most-suitable service provider 120 for the patient (e.g., based on the outcome predictions). In some embodiments, the referral response 130 includes a suggested response (e.g., whether to accept or decline the referral for a specific service provider 120). In some embodiments, the referral response 130 indicates one or more changes that, if implemented by a given service provider 120, would make that service provider 120 a better fit for the patient 105 or otherwise place them in a better position to accept referrals for similar patients.

In some embodiments, the referral prediction system 125 outputs the referral response 130 to a user, such as a healthcare provider who is making the referral and/or a healthcare provider who receives and evaluates the referral. This allows the user to determine whether to accept or decline the referral. In some embodiments, the referral prediction system 125 may itself accept and/or decline referrals (e.g., by comparing the predicted outcomes to various criteria, such as threshold probabilities of adverse events occurring to the patient 105). In these ways, as discussed above, the referral prediction system 125 can substantially improve healthcare service provisioning, reduce resource waste, and reduce computational expense of the referring process.

### Example Workflow for Generating Predicted Outcomes for Patient Referrals

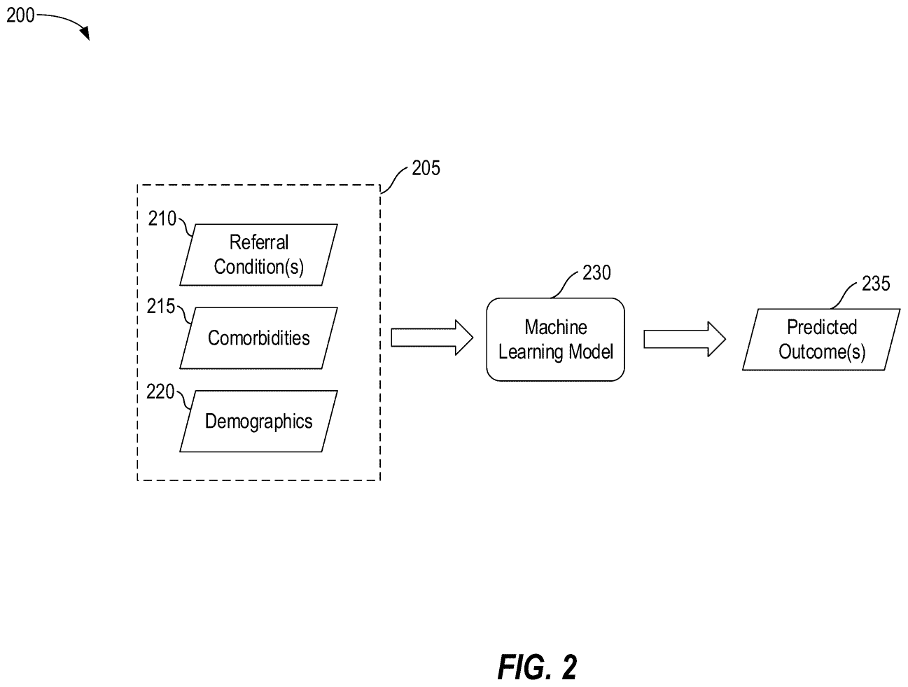

FIG. 2 depicts an example workflow 200 for generating predicted outcomes for patient referrals, according to one embodiment of the present disclosure. In some embodiments, the workflow 200 is performed by a referral prediction system, such as the referral prediction system 125 of FIG. 1.

In the illustrated example, patient data 205 is accessed by a machine learning model 230 to generate one or more predicted outcomes 235. In some embodiments, the patient data 205 corresponds to the patient data 110 of FIG. 1. As discussed above, the patient data 205 generally includes any relevant information that may be used, by the machine learning model 230, to generate the predicted outcome(s) 235. For example, in the illustrated workflow 200, the patient data 205 includes one or more referral conditions 210, comorbidities 215, and demographics 220.

As discussed above, the referral condition(s) 210 generally correspond to the condition(s) for which the patient is being referred. In some aspects, the referral conditions 225 are indicated in the patient referral itself. For example, a doctor may write a referral for the patient, indicating that the patient should seek further treatment from a specialist for the indicated referral condition(s) 210. In some aspects, the referral condition(s) 210 may alternatively be referred to as referral diagnoses.

As discussed above, the comorbidities 215 may generally correspond to any other condition(s) or diagnoses that the patient has which may affect their outcomes. For example, comorbidities may include diagnoses such as high blood pressure, diabetes, and the like. In some embodiments, the comorbidities 215 include any diagnoses of the patient other than the referral conditions 210. In some embodiments, the comorbidities 215 may correspond to specific referral conditions 210 (e.g., where each referral condition 210 has one or more specific diagnoses listed or understood as comorbidities for the referral condition 210).

In some embodiments, the demographics 220 generally include any demographic information of the patient that may be useful in generating predicted outcomes 235. For example, as discussed above, the demographics 220 may include the patient's age, weight, sex, and the like. Although not depicted in the illustrated example, in some embodiments, the patient data 205 may include other data such as the medications the patient takes, allergies the patient has, and the like.

In the illustrated workflow 200, the machine learning model 230 processes the patient data 205 to generate the predicted outcomes 235. In some embodiments, as discussed above, the machine learning model 230 may process the patient data 205 in response to a patient referral (e.g., when a patient referral is received), in response to a user request (e.g., when a user who received a referral requests that outcomes be predicted, and/or when a user who is writing a referral asks that outcomes be predicted), and the like.

Although not depicted in the illustrated example, in some embodiments, the patient data 205 may undergo various preprocessing and/or feature extraction prior to being processed by the machine learning model 230. For example, the machine learning model 230 (or another component) may extract relevant or salient features, vectorize the data, and the like.

As discussed above, each predicted outcome 235 generally comprises a prediction relating to what will happen, to the patient being referred, if the referral to a given target service is accepted by the target service and the patient is enrolled or admitted to the service. For example, the predicted outcomes 235 may indicate a categorical prediction as to whether the patient will recover from the referral conditions 210, whether the referral conditions 210 will worsen, or whether the referral conditions 210 will remain the same. In some embodiments, the predicted outcomes 235 may include one or more predicted probabilities about the outcome (e.g., the probability that the patient will recover).

In some embodiments, the predicted outcomes 235 may include predictions about whether the patient will suffer any adverse events if the referral is accepted by the target provider, such as whether the patient will become septic, whether the patient will require hospitalization, and the like. In some embodiments, the predicted outcomes 235 may include timeline-related predictions, such as how much time will pass before the patient recovers, how much time will pass before the adverse event(s) occur, how much time will pass before the patient is deceased (in the case of a hospice referral), and the like.

Generally, the particular contents of the predicted outcomes 235 may vary depending on the particular implementation and referral. For example, the predicted outcomes 235 may comprise specific type(s) of predictions for each referral condition 210.

In some embodiments, each of the predicted outcomes 235 may be generated by a corresponding machine learning model 230. That is, the machine learning model 230 may be trained to predict a specific type of outcome (e.g., recovery probability) for a specific referral condition 210, while a second model may be trained to predict another outcome (e.g., adverse event probabilities). In some embodiments, therefore multiple models may be used to generate a set of predicted outcomes 235. In other embodiments, one or more of the models may be trained to predict multiple predicted outcomes 235.

In some embodiments, the predicted outcomes 235 may be service-specific. That is, each predicted outcome 235 may indicate a prediction if the patient is accepted into a specific healthcare service (e.g., a service provider 120 of FIG. 1). In some embodiments, the machine learning model 230 is similarly service-specific. For example, there may be a separate machine learning model 230 trained for each respective service provider (e.g., where each model is trained based on data from the corresponding service provider). In some embodiments, the machine learning model 230 may be service-agnostic. For example, the machine learning model 230 may be trained using data from multiple service providers. In some embodiments, to generate service-specific predictions using a service-agnostic model, the machine learning model 230 may receive, as input, an indication of a specific service provider (alongside the patient data 205) in order to generate a prediction for the specific provider.

As discussed above, in some embodiments, the predicted outcomes 235 may be used to facilitate referral decisions. For example, in some embodiments, the predicted outcomes 235 may be provided to a user (e.g., a healthcare provider with the referral target) to determine whether the referral should be accepted. In some embodiments, the predicted outcomes 235 may be evaluated automatically (e.g., by a referral prediction system) to generate a suggestion or recommendation (e.g., by comparing the predicted outcomes 235 to one or more criteria).

In some embodiments, the predicted outcomes 235 may be used to generate suggestions or recommendations, such as to identify another healthcare service that is better-suited to accept the referral (e.g., that will provide better outcomes), or to indicate changes that the target service could make to place them in a better position to accept the referral (or to accept other similar referrals), as discussed in more detail below.

### Example Workflow for Generating Predicted Outcomes and Revised Referrals for Patient Referrals

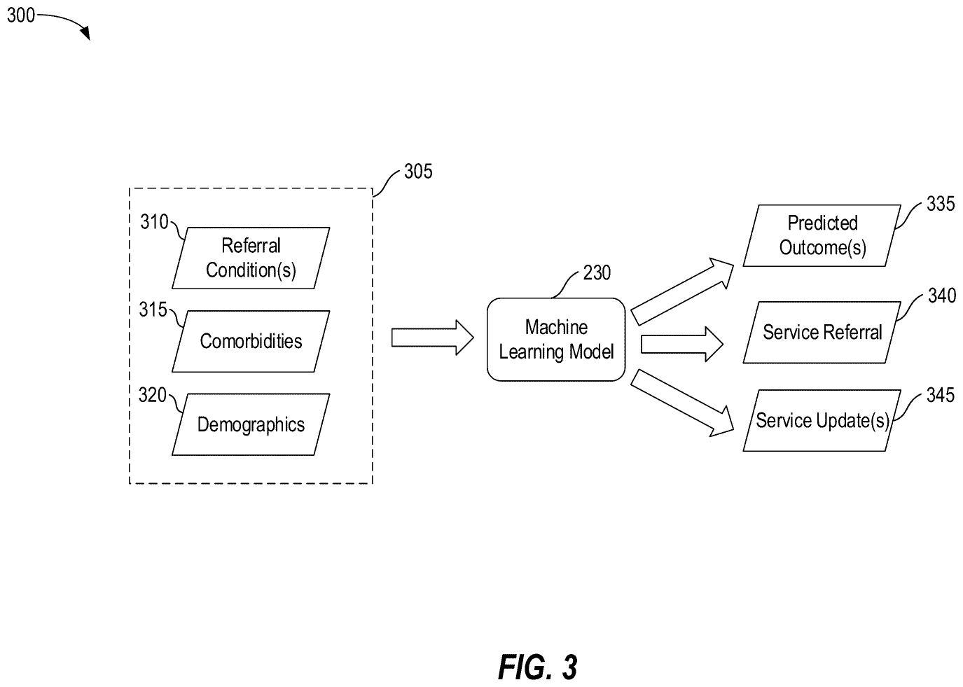

FIG. 3 depicts an example workflow 300 for generating predicted outcomes and revised referrals for patient referrals, according to one embodiment of the present disclosure. In some embodiments, the workflow 300 is performed by a referral prediction system, such as the referral prediction system 125 of FIG. 1.

In the illustrated example, patient data 305 is accessed by a machine learning model 230 to generate one or more predicted outcomes 335, as well as service referral 340 and service update(s) 345. In some embodiments, the patient data 305 corresponds to the patient data 110 of FIG. 1 and/or the patient data 205 of FIG. 2. As discussed above, the patient data 305 generally includes any relevant information that may be used, by the machine learning model 230, to generate the predicted outcome(s) 335, service referral 340, and/or service updates 345. For example, in the illustrated workflow 300, the patient data 305 includes one or more referral conditions 310, comorbidities 315, and demographics 320.

As discussed above, the referral condition(s) 310 generally correspond to the condition(s) for which the patient is being referred. The comorbidities 315 may generally correspond to any other condition(s) or diagnoses that the patient has which may affect their outcomes, and the demographics 320 may generally include any demographic information of the patient that may be useful in generating predicted outcomes 335, service referral 340, and/or service updates 345.

In the illustrated workflow 300, the machine learning model 230 processes the patient data 305 to generate the predicted outcomes 335. In some embodiments, as discussed above, the machine learning model 230 may process the patient data 305 in response to a patient referral (e.g., when a patient referral is received), in response to a user request (e.g., when a user who received a referral requests that outcomes be predicted, and/or when a user who is writing a referral asks that outcomes be predicted), and the like.

Although not depicted in the illustrated example, in some embodiments, the patient data 305 may undergo various preprocessing and/or feature extraction prior to being processed by the machine learning model 230. For example, the machine learning model 230 (or another component) may extract relevant or salient features, vectorize the data, and the like.

As discussed above, each predicted outcome 335 generally comprises a prediction relating to what will happen, to the patient being referred, if the referral to a given target service is accepted by the target service and the patient is enrolled or admitted to the service. For example, the predicted outcomes 335 may indicate a categorical prediction as to whether the patient will recover from the referral conditions 310, whether the referral conditions 310 will worsen, or whether the referral conditions 310 will remain the same. In some embodiments, the predicted outcomes 335 may include one or more predicted probabilities about the outcome (e.g., the probability that the patient will recover). Generally, the particular contents of the predicted outcomes 335 may vary depending on the particular implementation and referral.

As discussed above, in some embodiments, each of the predicted outcomes 335 may be generated by a corresponding machine learning model 230 (e.g., the machine learning model 230 may be trained to predict a specific type of outcome for a specific referral condition 310). That is, multiple models may be used to generate a set of predicted outcomes 335.

In some embodiments, as discussed above, the predicted outcomes 335 may be service-specific. That is, each predicted outcome 335 may indicate a prediction if the patient is accepted into a specific healthcare service (e.g., a service provider 120 of FIG. 1). As discussed above, in some embodiments, the predicted outcomes 335 may be used to facilitate referral decisions. For example, in some embodiments, the predicted outcomes 335 may be provided to a user (e.g., a healthcare provider with the referral target) to determine whether the referral should be accepted. In some embodiments, the predicted outcomes 335 may be evaluated automatically (e.g., by a referral prediction system) to generate a suggestion or recommendation (e.g., by comparing the predicted outcomes 335 to one or more criteria).

In the illustrated example, the machine learning model 230 is also used to generate an updated service referral 340, and one or more service updates 345. The service referral 340 generally comprises a suggestion, instruction, or recommendation that the patient be referred to one or more specific service providers. For example, in response to determining (based on the predicted outcomes 335) that the original or initial referral target is not well-suited to take on the patient, the system may identify or select another (better suited) provider to include in the service referral 340.

In some embodiments, as discussed above, the referral prediction system may evaluate multiple alternative service providers. In some embodiments, rather than evaluating multiple providers initially, the referral prediction system may first evaluate the actual referral target (e.g., the healthcare service that is actually specified in the initial referral) to generate predicted outcomes 335 for this target. In some embodiments, the referral prediction system only proceeds to evaluate alternative providers if the predicted outcomes 335 indicate that the initial target is not well-suited.

For example, the referral prediction system may use the machine learning model(s) 230 to generate predicted outcomes 335 for the referral target. If the predicted outcomes 335 fail to meet defined criteria (e.g., the probability of the patient's condition worsening is high), the referral prediction system may then determine to use the machine learning model(s) 230 to generate new predicted outcomes for one or more alternative healthcare service providers. This can substantially reduce the computational expense, as the model(s) need only be used to process data once (for the referral target) unless the target is ill-suited. In that case, the models may be used again (incurring additional computational expense) to process alternatives. By using such an iterative approach, the referral prediction system may reduce the computational expense of processing input data to evaluate referrals.

In some embodiments, the service referral 340 corresponds to or indicates a healthcare service that is relatively better suited to accept the patient, as compared to the referral target, based on corresponding predicted outcomes 335. For example, the service referral 340 may indicate which service alternative is best suited (e.g., because it has the best predicted outcomes for the patient). In some aspects, if the referral target is the best-suited option, the service referral 340 may indicate the next-best fit (e.g., the healthcare service having the next-best predicted outcomes for the patient).

In these ways, the referral prediction system facilitates routing of the patient to an appropriate healthcare service based on predicted outcomes. In some aspects, the service referral 340 is output (e.g., to a user, such as a doctor) to review. If the user agrees with the referral (e.g., based on the predicted outcomes at the referral target and/or indicated alternative), the user may provide the referral to the patient and/or the new target.

In some embodiments, the service update(s) 345 indicate mutable characteristics or attributes of the referral target that, if changed, may improve the predicted outcomes for the patient. For example, as discussed above, the service updates 345 may indicate or suggest actions, such as hiring additional staff, which may improve patient outcomes. In some embodiments, to generate the service updates 345, the referral prediction system may compare the characteristics of the referral target to the characteristics of one or more other service providers. For example, in response to determining that an alternative provider is better suited (e.g., with better predicted outcomes 335), the referral prediction system may identify difference(s) between this better alternative and the original referral target.

In some embodiments, once such differences are identified, they may be indicated as service updates 345, which may be output to a user for review (e.g., to an administrator or clinician at the referral target). In some embodiments, in addition to or instead of identifying service differences based on one patient, the referral prediction system can aggregate this analysis based on multiple patients. For example, the referral prediction system may track or monitor referrals that are declined and/or referrals that are accepted but where the patient has poor outcomes, in order to determine or identify commonalities between these patients. Once such commonalities are identified, the referral prediction system may similarly identify service updates 345 that would better position the referral target to care for these patients.

This can substantially improve the efficacy and quality of healthcare provided, while ensuring more efficient and targeted use of resources across systems.

### Example Workflow for Generating Predicted Outcomes for Patient Referrals and Refining Machine Learning Models Based on Subsequent Outcome Feedback

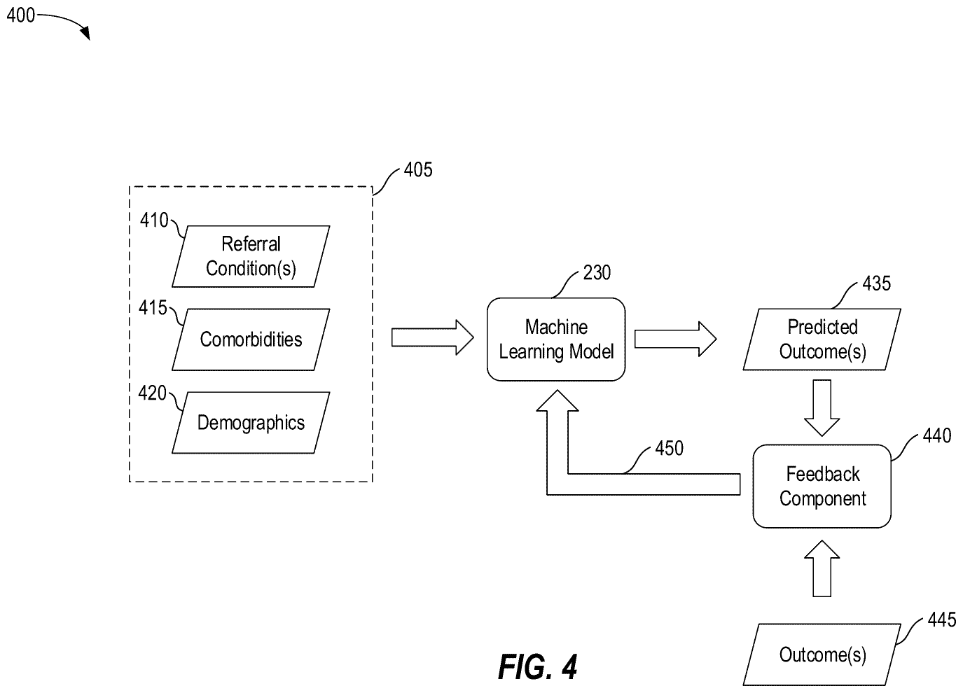

FIG. 4 depicts an example workflow 400 for generating predicted outcomes for patient referrals and refining machine learning models based on subsequent outcome feedback, according to one embodiment of the present disclosure. In some embodiments, the workflow 400 is performed by a referral prediction system, such as the referral prediction system 125 of FIG. 1.

In the illustrated example, patient data 405 is accessed by a machine learning model 230 to generate one or more predicted outcomes 435. In some embodiments, the patient data 405 corresponds to the patient data 110 of FIG. 1, the patient data 205 of FIG. 2, and/or the patient data 305 of FIG. 3. As discussed above, the patient data 405 generally includes any relevant information that may be used, by the machine learning model 230, to generate the predicted outcome(s) 435. For example, in the illustrated workflow 400, the patient data 405 includes one or more referral conditions 410, comorbidities 415, and demographics 420.

As discussed above, the referral condition(s) 410 generally correspond to the condition(s) for which the patient is being referred. The comorbidities 415 may generally correspond to any other condition(s) or diagnoses that the patient has which may affect their outcomes, and the demographics 420 may generally include any demographic information of the patient that may be useful in generating predicted outcomes 435.

In the illustrated workflow 400, the machine learning model 230 processes the patient data 405 to generate the predicted outcomes 435. In some embodiments, as discussed above, the machine learning model 230 may process the patient data 405 in response to a patient referral (e.g., when a patient referral is received), in response to a user request (e.g., when a user who received a referral requests that outcomes be predicted, and/or when a user who is writing a referral asks that outcomes be predicted), and the like.

Although not depicted in the illustrated example, in some embodiments, the patient data 405 may undergo various preprocessing and/or feature extraction prior to being processed by the machine learning model 230. For example, the machine learning model 230 (or another component) may extract relevant or salient features, vectorize the data, and the like.

As discussed above, each predicted outcome 435 generally comprises a prediction relating to what will happen, to the patient being referred, if the referral to a given target service is accepted by the target service and the patient is enrolled or admitted to the service. For example, the predicted outcomes 435 may indicate a categorical prediction as to whether the patient will recover from the referral conditions 410, whether the referral conditions 410 will worsen, or whether the referral conditions 410 will remain the same. In some embodiments, the predicted outcomes 435 may include one or more predicted probabilities about the outcome (e.g., the probability that the patient will recover). Generally, the particular contents of the predicted outcomes 435 may vary depending on the particular implementation and referral.

In the illustrated workflow 400, after a referral is accept or declined (e.g., based on the predicted outcomes 435), a feedback component 440 may monitor the patient's healthcare progress to determine how well the predicted outcomes 435 align with real outcomes 445. For example, if the patient is accepted to the referral target, the feedback component 440 may evaluate the actual patient outcomes 445 at the referral target, and compare these outcomes to the predicted outcomes 435. Similarly, if the patient is declined by the referral target but accepted at a second service provider, the feedback component 440 may compare the outcomes 445 at this provider with the predicted outcomes 435 for the second provider.

In some embodiments, the feedback component 440 determines the outcomes 445 after one or more defined criteria are satisfied, such as a defined time period, the occurrence of a defined event, and the like. For example, in some embodiments, the feedback component 440 may determine whether the patient has been discharged or released from the healthcare service. If so, the feedback component 440 may determine the outcomes 445. (e.g., the state of the referral condition(s) 410 (such as whether the patient recovered or declined), the time that elapsed between the referral being accepted and the discharge, whether the patient was hospitalized or had any other adverse events during treatment, and the like).

Generally, the particular outcomes 445 evaluated may depend on the particular criteria and/or referral. For example, if the feedback component 440 determines that the patient has been hospitalized while being treated by the healthcare service, the feedback component 440 may use this event as an outcome 445 to compare against the predicted outcomes 435.

In the illustrated example, based on comparing the predicted outcomes 435 and the outcomes 445, the feedback component 440 can update the machine learning model(s) 230, as illustrated by arrow 450. Generally, the specific techniques used to update the machine learning models 230 may vary depending on the particular implementation and architecture. For example, if the machine learning model 230 is a neural network architecture, the feedback component 440 may generate a loss based on the predicted outcomes 435 and actual outcomes 445, and update the model based on backpropagating the loss through the network. Generally, the particular loss formulation may vary depending on the particular implementation. For example, for categorical or probability predictions (e.g., predicting the likelihood that the patient will recover), the feedback component 440 may use a cross-entropy loss formulation. As another example, for regression predictions (e.g., predicting the time that will elapse until an event occurs), the feedback component 440 may use a mean squared error loss formulation.

In this way, the machine learning model 230 can be updated during inferencing, enabling the referral prediction system to continue to generate highly accurate predictions using continuously updated models. In some embodiments, the referral prediction system may update the machine learning model 230 periodically (e.g., the losses may be aggregated or stored until the periodic updating time arrives, at which point the model may be updated based on all generated losses). This may allow the referral prediction system to update the models during off-peak times, such as overnight, when computational load on the system is minimized. In this way, the referral prediction system can more efficiently provide continuous training of the models without harming other ongoing workloads (such as inferencing itself), improving the functionality and operations of the referral prediction system and the model.

### Example Workflow for Training Machine Learning Models to Evaluate Patient Referrals

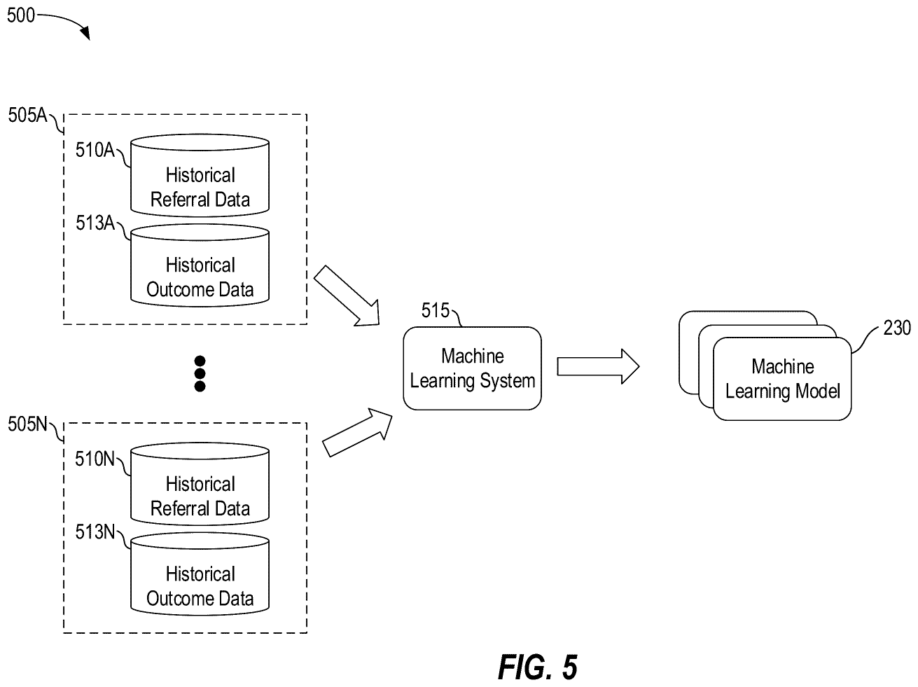

FIG. 5 depicts an example workflow 500 for training machine learning models to evaluate patient referrals, according to one embodiment of the present disclosure. In some embodiments, the workflow 500 is performed by a referral prediction system, such as the referral prediction system 125 of FIG. 1 (e.g., the machine learning system 515 may correspond to the referral prediction system 125 of FIG. 1). That is, referral prediction system may perform both model training as well as inferencing using trained models. In other embodiments, the machine learning system 515 is a separate system. That is, one system may be used to train the models, while another system is used to inference based on the trained models.

In the illustrated workflow 500, sets of training data 505A-N (collectively, training data 505) are accessed by a machine learning system 515, which trains a set of one or more machine learning models 230. In the illustrated example, two distinct sets of training data 505 are depicted. In embodiments, there may be any number and variety of sets of training data 505.

In some embodiments, each set of training data 505 corresponds to a respective healthcare service provider (e.g., a service provider 120 of FIG. 1). For example, the training data 505A may correspond to a first service provider, while the training data 505N may correspond to a second service provider. That is, the training data 505A may include data that is associated with and/or received from the first service provider (e.g., based on patients that the first service provider treated and/or accepted referrals for), while the training data 505N may include data associated with and/or received from the second service provider (e.g., based on patients that the second service provider treated). By using this service-specific training data 505, the machine learning system 515 may train machine learning models 230 that can generate service-specific predictions, as discussed below in more detail.

In the illustrated example, each set of training data 505 comprises a set of historical referral data 510 and a set of historical outcome data 513. Specifically, the training data 505A comprises historical referral data 510A and historical outcome data 513A, while the training data 505N comprises historical referral data 510N and historical outcome data 513N. In some embodiments, the historical referral data 510 generally comprises information about prior patient referrals to the corresponding healthcare service. That is, the historical referral data 510 includes information about patients that were referred to the healthcare service. In some embodiments, the historical referral data 510 only includes information about referrals that were accepted (e.g., declined referrals may be excluded).

In some embodiments, the historical referral data 510 corresponds to or comprises patient data about referrals that were accepted by the provider. For example, the historical referral data 510 may comprise patient data 110 of FIG. 1, patient data 205 of FIG. 2, patient data 305 of FIG. 3, and/or patient data 405 of FIG. 4. In some embodiments, the historical referral data 510 includes a set of records (also referred to as a sample and/or an exemplar), where each record comprises patient data about a patient that was accepted to the healthcare service. For example, each record may include information such as the patient's referral condition(s), comorbidities, demographics, allergies, medication use, and the like. In some embodiments, the historical referral data 510 includes any features or information that is (or will be) used to generate input to the machine learning models during inferencing.

In some embodiments, the historical referral data 510 includes information as of the time of the referral. That is, rather than including the current or up-to-date information for each patient, the historical referral data 510 may reflect the patient data as of the time when the referral was made and/or accepted. In some embodiments, the information in the historical referral data 510 is used as the input portion of each training exemplar, as discussed in more detail below.

In the illustrated example, the historical outcome data 513 generally corresponds to or comprises information about outcome(s) experienced by patients that were accepted and/or treated by the corresponding healthcare service. For example, the historical outcome data 513 may correspond to outcomes 445 of FIG. 4. As discussed above, the particular information included in the historical outcome data 513 may vary depending on the particular implementation, and may include information such as whether the patient recovered from their referral condition(s), the time that elapsed before discharge, any adverse events that occurred, and the like.

In some embodiments, the historical outcome data 513 includes a set of records, where each record comprises outcome information for a patient. In some embodiments, there is a one-to-one mapping between the historical referral data 510 and historical outcome data 513 with respect to each set of training data 505. That is, each training exemplar may comprise a pair of records: one from the historical referral data 510, and one from the historical outcome data 513 for the same patient.

For example, for each record in the historical referral data 510A, there may be a corresponding record in the historical outcome data 513A (and vice versa), each corresponding to the same patient. Similarly, for each record in the historical referral data 510N, there may be a corresponding record in the historical outcome data 513N (and vice versa), each corresponding to the same patient. In some embodiments, the information in the historical outcome data 513 is used as the target output or label portion of each training exemplar, as discussed in more detail below.

In the illustrated example, the machine learning system 515 uses the sets of training data 505 to train one or more machine learning models 230. In some embodiments, as discussed above, the machine learning system 515 trains service-specific machine learning models 230. That is, for each healthcare service, the machine learning system 515 may train a corresponding set of one or more service-specific models based on a corresponding set of training data 505. For example, the machine learning system 515 may use the training data 505A (from a first healthcare service) to train a first set of machine learning models 230 for the first healthcare service, and use the training data 505N (from a second healthcare service) to train a second set of machine learning models 230 for the second healthcare service.

By training each machine learning model 230 based only on data from a corresponding healthcare service, the machine learning system 515 can create specialized models that can be used to generate outcome predictions for the corresponding service. For example, during runtime, the machine learning system 515 may determine which healthcare service is the referral target, and use the corresponding set of model(s) to process the patient data.

In some embodiments, rather than training a separate set of models for each healthcare service, the machine learning system 515 may use the identity of each service as a separate input to the models during training. For example, the machine learning system 515 may train a unified or global set of machine learning models 230 based on all sets of training data 505. In some embodiments, during such training, the machine learning system 515 may include an indication of the healthcare service (e.g., a unique identifier) that corresponds to each exemplar. For example, when processing or training based on an exemplar from the training data 505A, the machine learning system 515 may use a unique identifier of the first healthcare service (in addition to patient data from the historical referral data 510) as input to the model.

By training the machine learning models 230 based on data across services, the machine learning system 515 can create generalized models. Nevertheless, these models may still be used to generate service-specific predictions, such as by providing the unique identifier of the referral target as input during inferencing (alongside patient data).

In some embodiments, the machine learning system 515 may train machine learning models 230 that are condition-specific. For example, for a first referral condition, the machine learning system 515 may train a first machine learning model 230 (based on training data 505 corresponding to the first referral condition). For a second referral condition, the machine learning system 515 may train a second machine learning model 230 (based on training data 505 corresponding to the second referral condition), and so on. By training each machine learning model 230 based only on data relevant to a corresponding referral condition, the machine learning system 515 can create specialized models that can be used to generate outcome predictions for the corresponding condition. For example, during runtime, the machine learning system 515 may determine which condition(s) are the cause of the referral (e.g., for what the patient is being referred), and use the corresponding set of model(s) to process the patient data.

In some embodiments, rather than training a separate set of models for each referral condition, the machine learning system 515 may use the identity of each condition as a separate input to the models during training. For example, the machine learning system 515 may train a unified or global set of machine learning models 230 based on all sets of training data 505. In some embodiments, during such training, the machine learning system 515 may include an indication of the referral condition (e.g., a unique identifier) that corresponds to each exemplar. For example, when processing or training based on an exemplar corresponding to a first referral condition, the machine learning system 515 may use a unique identifier of the first condition (in addition to patient data from the historical referral data 510) as input to the model. By training the machine learning models 230 based on data across conditions, the machine learning system 515 can create generalized models. Nevertheless, these models may still be used to generate condition-specific predictions, such as by providing the unique identifier of the referral condition as input during inferencing (alongside patient data).

In some embodiments, the machine learning system 515 may train machine learning models 230 that are outcome-specific. For example, for a first outcome, the machine learning system 515 may train a first machine learning model 230 (based on training data 505 corresponding to the first outcome). For a second outcome, the machine learning system 515 may train a second machine learning model 230 (based on training data 505 corresponding to the second outcome), and so on. By training each machine learning model 230 based only on data relevant to a corresponding outcome, the machine learning system 515 can create specialized models that can be used to generate outcome-specific predictions for the corresponding outcome. For example, during runtime, the machine learning system 515 may use a set of models to generate corresponding predictions for each relevant outcome (e.g., categorical and/or continuous value predictions for each outcome).

In some embodiments, rather than training a separate set of models for each outcome, the machine learning system 515 may train a model to predict multiple outcomes simultaneously, and/or may use the identity of each outcome as a separate input to the models during training. For example, the machine learning system 515 may train a unified or global set of machine learning models 230 based on all sets of training data 505. In some embodiments, during such training, the machine learning system 515 may include an indication of the relevant outcome (e.g., a unique identifier of the outcome for which the system would like to generate a prediction).

Generally, training the machine learning models 230 includes updating one or more parameters (e.g., weights) of the models based on the training data 505. For example, the parameters may be initialized (e.g., to random values), and the machine learning system 515 may use the training data 505 to iteratively learn values for the parameter(s) that enable improved predictions. The particular operations used by the machine learning system 515 to train the machine learning models 230 may vary depending on the particular implementation and architecture.

For example, if the machine learning models 230 use neural network architectures, the machine learning system 515 may process the input portion of a given training record (e.g., the historical referral data 510) as input to the model to generate an output (e.g., one or more predicted outcomes). In some aspects, as discussed above, a unique identifier of the healthcare service to which the training record corresponds and/or a unique identifier of the referral condition may also be used as input to generate the predicted outcome(s). The machine learning system 515 may then compare the generated output with the label portion of the record (e.g., the historical outcome data 513) to generate a loss (e.g., using cross entropy, mean squared error, and the like). This loss can then be used to update the parameters of the model, such as via backpropagation.

Generally, the machine learning system 515 repeat this process for each training exemplar across one or more iterations or epochs of training. For example, each epoch may correspond to a single pass of each record through the models (where other aspects, such as training hyperparameters, may change between epochs).

In an embodiment, once the machine learning models 230 are fully trained, the machine learning system 515 may deploy them for inferencing. As discussed below in more detail, the machine learning system 515 may use a variety of termination criteria to determine when to deploy the models. For example, the machine learning system 515 may deploy the models after a defined number of iterations or epochs have been performed, after no additional training data 505 remains for processing, after the models have reached a preferred accuracy level, and the like.

As discussed below in more detail, deploying the machine learning models 230 can generally include any operations needed to prepare or provide them for inferencing. For example, the machine learning system 515 may compile or package the learned parameters and architecture, transmit the model(s) to one or more inferencing systems, deploy the model(s) for local inferencing (e.g., allocating memory space), and the like.

### Example Method for Evaluating Referrals Using Machine Learning

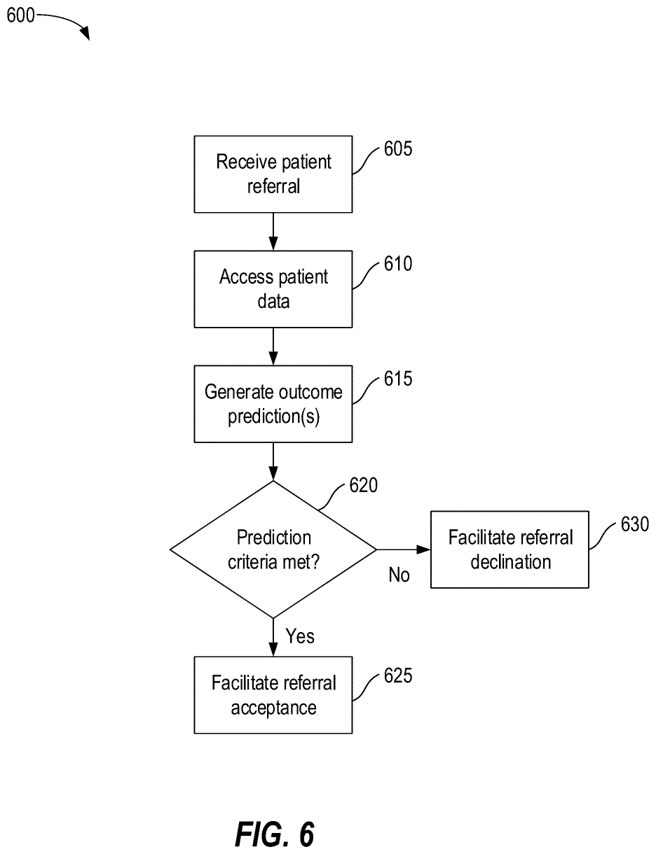

FIG. 6 is a flow diagram depicting an example method 600 for evaluating referrals using machine learning, according to one embodiment of the present disclosure. In some embodiments, the method 600 is performed by a referral prediction system, such as the referral prediction system 125 of FIG. 1.

At block 605, the referral prediction system receives a patient referral. For example, as discussed above, the patient referral may be created by a first healthcare provider (e.g., a clinician) to instruct that the patient seek treatment from an indicated healthcare service (e.g., a specialist). In some aspects, as discussed above, the patient referral identifies the patient (e.g., using a unique identifier) and at least one referral target (e.g., the service provider, such as service provider 120 of FIG. 1, that the patient should seek treatment from). In some embodiments, the patient referral may include other information such as identifying the referral condition(s).

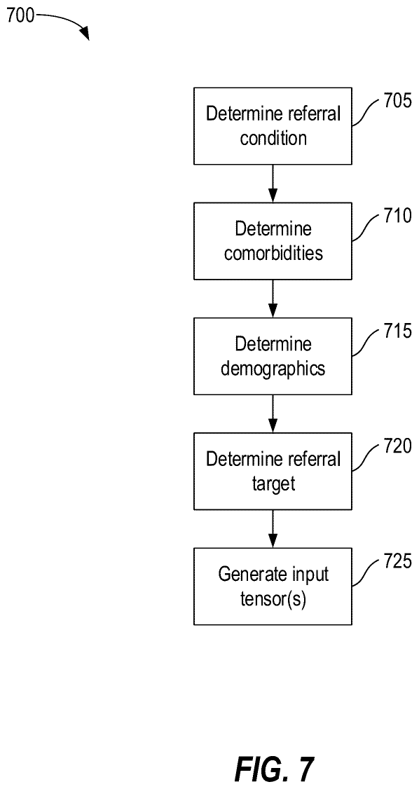

At block 610, the referral prediction system accesses patient data corresponding to the patient associated with the received referral. For example, the patient data may be included in the referral, or may be accessed from one or more other repositories (e.g., based on the patient identifier). The patient data may generally include any information used as input to the machine learning model(s), such as the referral condition, comorbidities, allergies, medications, demographics, and the like. For example, the patient data may correspond to the patient data 110 of FIG. 1, the patient data 205 of FIG. 2, the patient data 305 of FIG. 3, and/or the patient data 405 of FIG. 4. One example method to access the patient data is discussed in more detail below with reference to FIG. 7.

At block 615, the referral prediction system generates one or more outcome predictions for the patient based on processing some or all of the patient data using one or more machine learning models. In some embodiments, the referral prediction system may first perform feature extraction to extract or generate salient features based on the patient data, as discussed above. These features may then be used as input to the model(s). The predicted outcomes may generally correspond to any relevant outcome of interest, with respect to the referral, such as the probability of recovery, the probability of adverse events, and the like. In some embodiments, the predicted outcomes include a prediction as to how long the patient will receive or need treatment by the referral target, how much resources (e.g., healthcare resources) will be expended during such treatment, the predicted monetary cost or value of such treatment, and the like.

In some embodiments, the outcome predictions correspond to the predicted outcomes 235 of FIG. 2, the predicted outcomes 335 of FIG. 3, and/or the predicted outcomes 435 of FIG. 4. In some embodiments, the model(s) used to generate the outcome predictions may correspond to the machine learning model(s) 230 of FIGS. 2-5.

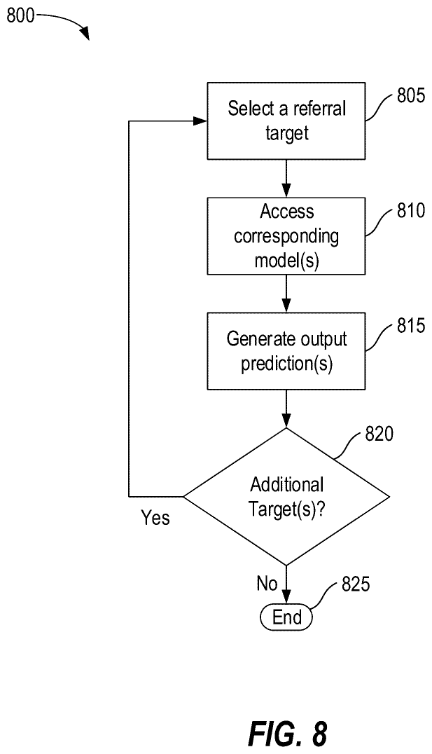

The particular operations used to generate the outcome predictions may vary depending on the particular implementation, and may include using one or more service-specific models for the referral target, one or more condition-specific models for the referral condition, and/or one or more outcome-specific models for the relevant outcome predictions. This may include use of models specific to a combination of such factors, such as a model specific to a given condition, service, and/or outcome. Example methods to generate the outcome predictions are discussed in more detail below with reference to FIGS. 8, 9, and 11.

At block 620, the referral prediction system determines whether one or more prediction criteria are satisfied. The particular prediction criteria may vary depending on the particular implementation. Generally, the prediction criteria can include any considerations or evaluations performed to determine whether the patient referral should be accepted by the referral target. For example, the referral prediction system may determine whether the referral target is a good fit for the patient at the time. In some embodiments, the referral prediction system may evaluate the outcome predictions using a set of rules, such as to determine whether the probability of recovery meets or exceeds a threshold, to determine whether the probability of adverse events is below a threshold (or is at least comparable to the probability of adverse events if the patient is treated by another facility), to compare the predicted resource usage and/or costs of treatment to one or more thresholds, and the like.

In some aspects, rather than the referral prediction system evaluating prediction criteria, the referral prediction system may instead output the outcome predictions (e.g., for a user to review). That is, the referral prediction system may allow the user to evaluate the predictions, rather than doing so directly. This may reduce computational expense of the method 600.

If, at block 620, the referral prediction system determines that the prediction criteria are not met, the method 600 continues to block 630, where the referral prediction system facilitates declination of the referral. That is, the referral prediction system facilitates the process of declining the referral, such as by outputting the outcome predictions and/or referral recommendation to a user to review and approve. Generally, facilitating the declination of the referral may include a variety of operations, such as declining the referral automatically, suggesting to a user that the referral be declined, outputting the predictions to a user and allowing the user to accept or decline the referral, and the like. In some embodiments, as discussed above and below in more detail, the referral prediction system may additionally or alternatively generate revised referral recommendations, service change recommendations, and the like.

Returning to block 620, if the referral prediction system determines that the prediction criteria are met, the method 600 continues to block 625, where the referral prediction system facilitates acceptance of the referral. That is, the referral prediction system facilitates the process of accepting the referral, such as by outputting the outcome predictions and/or referral recommendation to a user to review and approve. Generally, facilitating the acceptance of the referral may include a variety of operations, such as accepting the referral automatically, suggesting to a user that the referral be accepted, outputting the predictions to a user and allowing the user to accept or decline the referral, and the like.

In some embodiments, in addition to facilitating acceptance of the referral, the referral prediction system may perform one or more additional actions to facilitate intake and/or treatment of the patient. As an example, in some embodiments, based on the predicted outcome(s), the referral prediction system may initiate one or more preventative or proactive actions. For example, if the prediction(s) indicate that the patient may develop a complication (e.g., sepsis), the referral prediction system may initiate one or more proactive measures, such as notifying caregiver(s) to watch closely for sepsis. As another example, the referral prediction system may ensure that adequate supplies and training are available in the event that the patient becomes septic. For example, the referral prediction system may evaluate the current inventory of treatment supplies, evaluate current expertise of the caregiver(s), and assign specific caregiver(s) to the patient based on their expertise with respect to the predicted outcome(s) (e.g., assigning caregivers who are experienced with identifying and/or treating sepsis). As another example, the referral prediction system may assign training or instruction sessions for the caregiver(s) to ensure that they are up-to-date with respect to treating the predicted outcome(s).

In some embodiments, therefore, the referral prediction system can facilitate or provide improved treatment to patients who are accepted and admitted to the facility. For example, these proactive treatment operations can reduce negative outcomes, such as by ensuring that the predicted problems are identified quickly (e.g., such that caregivers can identify sepsis earlier than they otherwise would have, because they are specifically watching closely for such symptoms), and/or that patients are treated more readily (e.g., because they are caught sooner and the caregivers are better trained with supplies more readily available), and the like.

### Example Method for Determining Relevant Patient Data for Evaluating Referrals

FIG. 7 is a flow diagram depicting an example method 700 for determining relevant patient data for evaluating referrals, according to one embodiment of the present disclosure. In some embodiments, the method 700 is performed by a referral prediction system, such as the referral prediction system 125 of FIG. 1. In some embodiments, the method 700 provides additional detail for block 610 of FIG. 6.

At block 705, the referral prediction system determines the referral condition(s) for the patient referral. For example, as discussed above, the referral conditions may be specified in the referral itself. In some embodiments, the referral prediction system may infer the referral conditions. In some such embodiments, the referral prediction system may evaluate patient data associated with the patient to infer the referral condition(s). For example, based on the referral target, the referral prediction system may infer the condition (e.g., inferring that the referral condition is cancer when the referral target is an oncologist). As another example, based on recent diagnoses (or recently worsened conditions) reflected in the patient data, the referral prediction system may infer that the referral condition is the most-recent diagnosis and/or a condition that recently worsened for the patient. In some embodiments, the referral condition corresponds to the referral condition 210 of FIG. 2, the referral condition 310 of FIG. 3, and/or the referral condition 410 of FIG. 4.

At block 710, the referral prediction system determines any comorbidities of the patient, with respect to the referral condition. For example, as discussed above, the referral prediction system may determine any other diagnoses that the patient has, and/or any other diagnoses that are relevant to the referral condition(s). In some embodiments, the comorbidities may be specified in the referral itself. In other embodiments, the referral prediction system may evaluate patient data associated with the patient to identify the comorbidities. In some embodiments, the comorbidities correspond to the comorbidities 215 of FIG. 2, the comorbidities 315 of FIG. 3, and/or the comorbidities 415 of FIG. 4.

At block 715, the referral prediction system determines any relevant demographics of the patient, with respect to the referral condition. For example, as discussed above, the referral prediction system may determine the patient's age, weight, ethnicity, sex, region where the patient lives, and the like. In some embodiments, the demographics may be specified in the referral itself. In other embodiments, the referral prediction system may evaluate patient data associated with the patient to identify the demographics. In some embodiments, the demographics correspond to the demographics 220 of FIG. 2, the demographics 320 of FIG. 3, and/or the demographics 420 of FIG. 4.

Although not included in the illustrated example, in some embodiments, the referral prediction system may similarly determine or access other relevant data, such as allergies that the patient has, medications that the patient uses, and the like.

At block 720, the referral prediction system determines the referral target for the patient referral. For example, as discussed above, the referral may specify which particular healthcare service the patient should seek treatment from. In some embodiments, if the referral does not uniquely identify a particular referral target, the referral prediction system may identify a set of possible alternative referral targets. For example, if the referral indicates that the patient should seek treatment for a skin condition (or if the primary care physician is preparing a referral but has not selected a specific target), the referral prediction system may identify healthcare services that are available (e.g., within a defined distance from the patient and accepting new patients) and are able to provide services for and/or treat the indicated referral condition (e.g., dermatologists in the area).

At block 725, the referral prediction system generates one or more input tensors for the machine learning models based on the determined data. For example, the referral prediction system may use feature extraction or other operations to generate a tensor encoding the referral condition(s), comorbidities, demographics, and/or referral target(s) (e.g., a tensor including a unique value for each input feature). This tensor can then be efficiently processed by the machine learning models, enabling improved outcome prediction.

### Example Method for Generating Predicted Outcomes Using Service-Specific Machine Learning Models

FIG. 8 is a flow diagram depicting an example method 800 for generating predicted outcomes using service-specific machine learning models, according to one embodiment of the present disclosure. In some embodiments, the method 800 is performed by a referral prediction system, such as the referral prediction system 125 of FIG. 1. In some embodiments, the method 800 provides additional detail for block 615 of FIG. 6.

At block 805, the referral prediction system selects a referral target. In some embodiments, selecting the referral target includes selecting or identifying the referral target indicated in the patient referral. In some embodiments, the referral prediction system may select the referral target from a set of alternative services, such as alternatives indicated in the referral itself, alternatives identified by the referral prediction system, and the like. In some embodiments, the referral prediction system may first evaluate the specific referral target(s) specified in the referral, and only selectively or dynamically evaluate other alternatives if the original target is determined to be a poor fit. Generally, if multiple referral targets are available or being evaluated, the referral prediction system may use any suitable techniques to select the target, including randomly or pseudo-randomly.

At block 810, the referral prediction system accesses the corresponding machine learning model(s) (e.g., machine learning models 230 of FIGS. 2-5) that were trained for the selected healthcare service (e.g., the selected referral target). For example, as discussed above, each model may be trained based on data specific to a given service, and the referral prediction system may use these service-specific models to generate service-specific outcome predictions for each referral target.

At block 815, the referral prediction system generates one or more output predictions based on processing the patient data using the accessed models that correspond to the selected referral target, as discussed above. For example, the referral prediction system may use the patient data as input to the model(s) to generate the output predictions.

At block 820, the referral prediction system determines whether there are any additional referral targets or alternatives that are yet-to-be evaluated. If so, the method 800 returns to block 805 to select a subsequent target. If not, the method 800 terminates at block 825. Although an iterative process (selecting and evaluating each healthcare service alternative separately) is depicted for conceptual clarity, in some embodiments, the referral prediction system may select and evaluate some or all of the alternative referral targets in parallel.

In this way, using service-specific models, the referral prediction system may generate more accurate and granular predictions with respect to each specific service, as compared to more generalized models.

### Example Method for Generating Predicted Outcomes Using Service-Agnostic Machine Learning Models

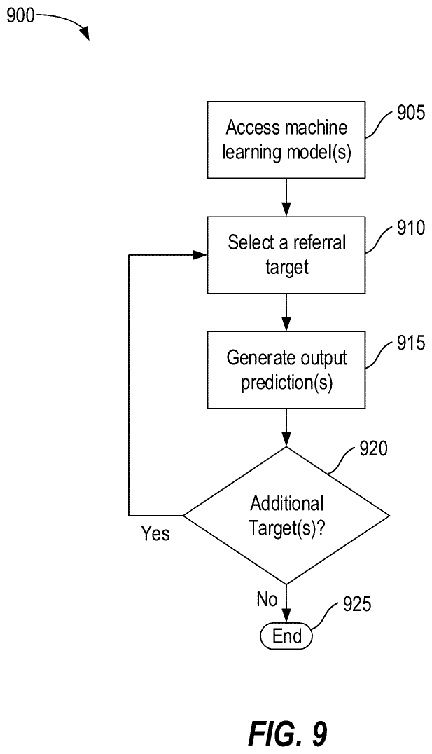

FIG. 9 is a flow diagram depicting an example method 900 for generating predicted outcomes using service-agnostic machine learning models, according to one embodiment of the present disclosure. In some embodiments, the method 900 is performed by a referral prediction system, such as the referral prediction system 125 of FIG. 1. In some embodiments, the method 900 provides additional detail for block 615 of FIG. 6.

At block 905, the referral prediction system accesses a set of trained machine learning model(s) (e.g., machine learning models 230 of FIGS. 2-5) that were trained to generate outcome predictions for multiple healthcare services. For example, as discussed above, each model may be trained based in part on the unique identifier of each given service, and the referral prediction system may use these service identifies to cause the service-agnostic model to generate service-specific outcome predictions for a specific referral target.

At block 910, the referral prediction system selects a referral target. In some embodiments, selecting the referral target includes selecting or identifying the referral target indicated in the patient referral. In some embodiments, the referral prediction system may select the referral target from a set of alternative services, such as alternatives indicated in the referral itself, alternatives identified by the referral prediction system, and the like. In some embodiments, the referral prediction system may first evaluate the specific referral target(s) specified in the referral, and only selectively or dynamically evaluate other alternatives if the original target is determined to be a poor fit. Generally, if multiple referral targets are available or being evaluated, the referral prediction system may use any suitable techniques to select the target, including randomly or pseudo-randomly.

At block 915, the referral prediction system generates one or more output predictions based on processing the patient data using the accessed models that correspond to the selected referral target, as discussed above. For example, the referral prediction system may use the patient data, in conjunction with the unique identifier of the selected referral target, as input to the model(s) to generate the output predictions.

At block 920, the referral prediction system determines whether there are any additional referral targets or alternatives that are yet-to-be evaluated. If so, the method 900 returns to block 910 to select a subsequent target. If not, the method 900 terminates at block 925. Although an iterative process (selecting and evaluating each healthcare service alternative separately) is depicted for conceptual clarity, in some embodiments, the referral prediction system may select and evaluate some or all of the alternative referral targets in parallel.

In this way, using service-agnostic models, the referral prediction system may generate outcome predictions with relatively reduced computational expense (e.g., because a smaller set of models need to be trained and stored or maintained), as compared to more specialized models.

### Example Method for Generating Proposed Healthcare Modifications to Improve Referral Outcomes

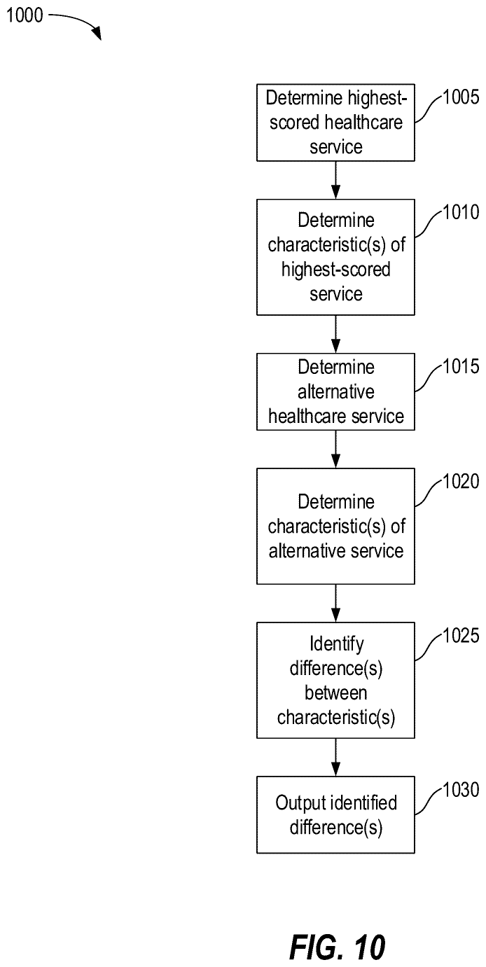

FIG. 10 is a flow diagram depicting an example method 1000 for generating proposed healthcare modifications to improve referral outcomes, according to one embodiment of the present disclosure. In some embodiments, the method 1000 is performed by a referral prediction system, such as the referral prediction system 125 of FIG. 1. In some aspects, the referral prediction system performs the method 1000 only if it is determined that the referral target (specified in the patient referral) may not be the best fit to accept and treat the patient.

At block 1005, the referral prediction system determines or identifies the highest-scored healthcare service. That is, the referral prediction system may identify which healthcare service has the best outcome predictions with respect to the patient (e.g., the highest probability of recovery, the lowest probability of adverse events, and the like). In some embodiments, the referral prediction system may generate scores for each healthcare service based on the outcome predictions for the service (e.g., by computing a weighted sum of the individual predictions for the service). This may allow for efficient comparison of service alternatives.

At block 1010, the referral prediction system determines one or more characteristics of the highest-scored service. For example, as discussed above, the referral prediction system may determine information that may affect patient outcomes, such as the number of staff, the staff mix (e.g., the percentage that are RNs), the staff-to-patient ratios, location, and the like.

At block 1015, the referral prediction system can similarly determine or identify at least one alternative healthcare service. For example, the referral prediction system may identify the referral target that was originally suggested in the referral.

At block 1020, the referral prediction system determines characteristic(s) of the alternative service. For example, as discussed above, the referral prediction system may determine information that may affect patient outcomes, such as the number of staff, the staff mix (e.g., the percentage that are RNs), the staff-to-patient ratios, location, and the like.

At block 1025, the referral prediction system identifies any differences or discrepancies between the services, with respect to the determined characteristics. For example, as discussed above, the referral prediction system may determine that the highest-scored service has a higher RN-to-patient ratio, a lower ratio of other staff members, different practices with respect to various procedures and operations, and the like.

At block 1030, the referral prediction system outputs the identified difference(s). For example, the referral prediction system may output service updates 345 of FIG. 3, suggesting that the referral target may be better suited to care for the patient (and other similarly situated patients) if the indicated changes are made.

In this way, the referral prediction system can substantially improve patient outcomes and enable more efficient resource allocations, improving the operations of the healthcare field.

### Example Method for Generating Predicted Outcomes Using Machine Learning

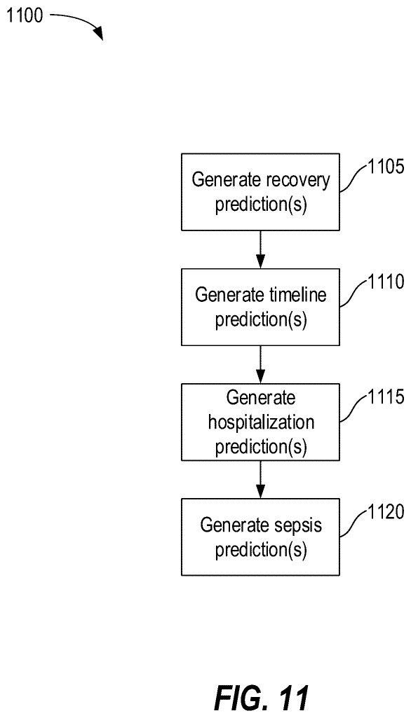

FIG. 11 is a flow diagram depicting an example method 1100 for generating predicted outcomes using machine learning, according to one embodiment of the present disclosure. In some embodiments, the method 1100 is performed by a referral prediction system, such as the referral prediction system 125 of FIG. 1. In some embodiments, the method 1100 provides additional detail for block 615 of FIG. 6.

At block 1105, the referral prediction system generates one or more recovery predictions based on processing the patient data using one or more machine learning models. The recovery predictions may generally include predictions about whether and how the patient conditions will change during treatment, if the referral target accepts the referral. For example, as discussed above, the referral prediction system may generate a probability that the patient will recover, a probability that the patient will experience adverse events, and the like.

At block 1110, the referral prediction system generates one or more timeline predictions based on processing the patient data using one or more machine learning models. The timeline predictions may generally include predictions about how much time will elapse before the patient conditions change during treatment, if the referral target accepts the referral. For example, as discussed above, the referral prediction system may generate a predicted amount of time that will elapse before recovery, a predicted amount of time that will elapse before one or more adverse events, and the like.

At block 1115, the referral prediction system generates one or more hospitalization predictions based on processing the patient data using one or more machine learning models. The hospitalization predictions may generally include predictions about whether and how the patient will need to be hospitalized during treatment, if the referral target accepts the referral. For example, as discussed above, the referral prediction system may generate a probability that the patient will require hospitalization.

At block 1120, the referral prediction system generates one or more sepsis predictions based on processing the patient data using one or more machine learning models. The sepsis predictions may generally include predictions about whether and how the patient will become septic, if the referral target accepts the referral. For example, as discussed above, the referral prediction system may generate a probability that the patient will become septic.

Although the illustrated example depicts a set of specific predictions, the referral prediction system may generally use machine learning to generate a wide variety of predictions relating to patient outcomes, depending on the particular implementation. In these ways, the referral prediction system can substantially improve patient outcomes by using machine learning to predict future outcomes for the patient.

### Example Method for Updating Machine Learning Models Based on Referral Outcomes

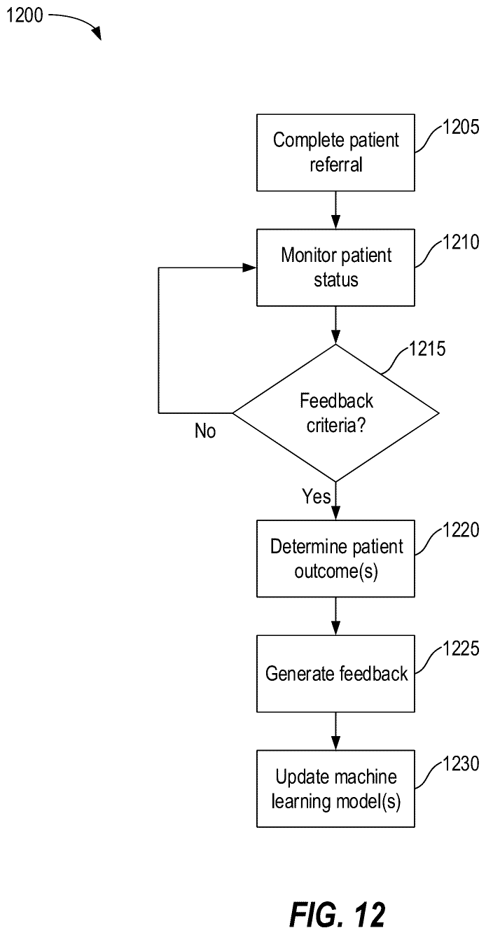

FIG. 12 is a flow diagram depicting an example method 1200 for updating machine learning models based on referral outcomes, according to one embodiment of the present disclosure. In some embodiments, the method 1200 is performed by a referral prediction system, such as the referral prediction system 125 of FIG. 1.

At block 1205, the referral prediction system completes a patient referral. For example, as discussed above, completing the referral may include evaluating it using machine learning (as discussed above) to generate outcome predictions. In some aspects, completing the referral includes facilitating acceptance or declination of the referral. For example, if the referral is accepted, the patient may be admitted or enrolled in the referral target service, and begin receiving treatment. If the referral is declined, the patient may subsequently be referred to and/or enrolled in an alternative healthcare service, receiving treatment from the alternative service.

At block 1210, the referral prediction system monitors the status of patient being treated by the target service and/or by an alternative service. For example, the referral prediction system may periodically determine whether the patient state has changed (e.g., whether they have recovered), whether the patient has experienced adverse events, and the like. In some embodiments, the referral prediction system may receive these updates using push operations. In other embodiments, the referral prediction system may request or retrieve these updates using pull operations.

At block 1215, the referral prediction system determines whether one or more feedback criteria are met. For example, based on the monitoring of the patient status, the referral prediction system may determine whether feedback related to patient outcomes is available to refine the machine learning model(s). If not, the method 1200 returns to block 1210.

If, at block 1215, the referral prediction system determines that feedback is available, the method 1200 continues to block 1220, where the referral prediction system determines the patient outcome(s) for the patient. For example, as discussed above, the referral prediction system may determine whether the patient recovered or worsened, the time that has elapsed since the treatment began, and the like. In some aspects, the outcomes correspond to the outcomes 445 of FIG. 4.

At block 1225, the referral prediction system generates feedback based on the outcomes. For example, the referral prediction system may preprocess the outcomes or otherwise format it in such a way that it can be used to train the model(s). For example, the referral prediction system may use the outcome(s) to generate one or more labels, and associate the label(s) with the corresponding patient data from the time when the referral was completed. In this way, the referral prediction system can dynamically generate new training exemplars whenever new outcome data is available.

At block 1230, the referral prediction system updates the machine learning model(s) based on the newly generated feedback. For example, as discussed above, the referral prediction system may update one or more service-specific models (for the healthcare service that accepted the referral), one or more condition-specific models (for the referral condition), and/or one or more outcome-specific models (for the outcome that has occurred).

As discussed above, the particular operations used to refine the models may vary depending on the particular implementation. For example, in the case of a neural network architecture, the referral prediction system may compare the actual outcome to the (original) predicted outcome to compute a loss, and use backpropagation to update the models based on the loss.

Although the illustrated example depicts updating of the model(s) using a single set of new feedback (e.g., using stochastic gradient descent), in some embodiments, the referral prediction system may instead store the feedback until a later time, and update the model(s) based on batches of samples (e.g., using batch gradient descent).

In these ways, the referral prediction system may continuously or periodically update the machine learning models during inferencing, enabling generation of highly accurate predictions.

### Example Method for Training Machine Learning Models to Predict Referral Outcomes

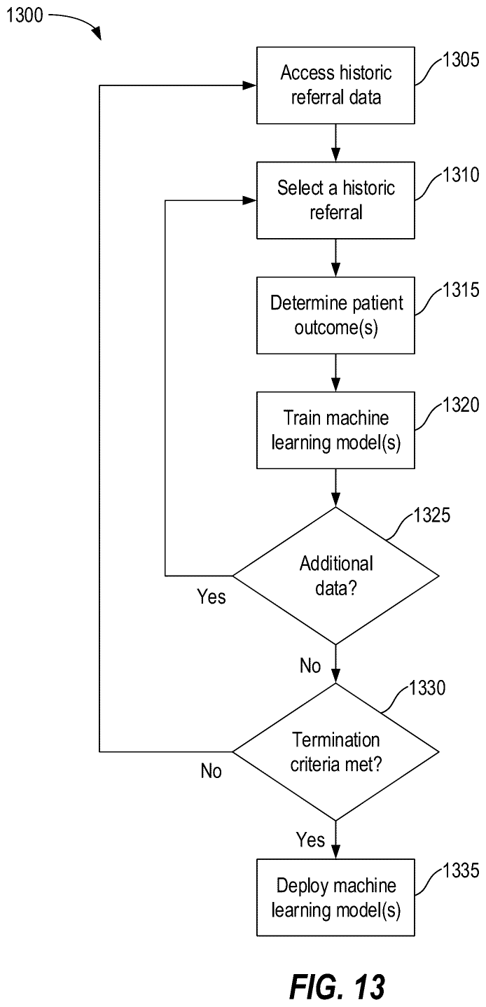

FIG. 13 is a flow diagram depicting an example method 1300 for training machine learning models to predict referral outcomes, according to one embodiment of the present disclosure. In some embodiments, the method 1300 is performed by a referral prediction system, such as the referral prediction system 125 of FIG. 1. In some embodiments, the method 1300 is performed by a machine learning system, such as the machine learning system 515 of FIG. 5.

At block 1305, the referral prediction system accesses a set of historic referral data. As discussed above, the historical referral data generally includes information for one or more prior patient referrals that were accepted by one or more healthcare services. For example, the historical referral data may include patient information such as their referral conditions, comorbidities, demographics, medications, allergies, and the like. In some embodiments, the historic referral data may correspond to the historic referral data 510 of FIG. 5.

At block 1310, the referral prediction system selects a historic referral from the set of historic referrals. That is, the referral prediction system may select a record or sample in the data, the record corresponding to a particular patient referral for a particular patient at a particular time. Generally, the referral prediction system may select the historic referral using any suitable technique, including randomly or pseudo-randomly, as all historic referrals may be processed during the method 1300.

At block 1315, the referral prediction system determines patient outcomes associated with the selected referral. That is, the referral prediction system may determine what outcome(s) were experienced by the patient, indicated in the patient referral, while the patient was being treated by the healthcare service. In some embodiments, for example, the referral prediction system may identify the corresponding data for the referral in the historical outcome data 513 of FIG. 5.

At block 1320, the referral prediction system trains one or more machine learning models based on the historic referral and patient outcomes. As discussed above, the particular operations used to train the models may vary depending on the particular implementation. For example, in the case of a neural network architecture, the referral prediction system may compare the actual outcome to a predicted outcome (generated by processing the patient data using the model) to compute a loss, and use backpropagation to update the models based on the loss.

Although the illustrated example depicts updating of the model(s) using a single historic referral (e.g., using stochastic gradient descent), in some embodiments, the referral prediction system may instead update the model(s) based on batches of referrals (e.g., using batch gradient descent).

In some embodiments, at block 1320, the referral prediction system may train one or more service-specific models (e.g., models trained specifically to generate predictions for the healthcare service that accepted the patient). In some embodiments, the referral prediction system may train one or more service-agnostic models (e.g., models trained to generate predictions for multiple service providers), such as by using a unique identifier of the healthcare service as input to the model. In some embodiments, the referral prediction system may train one or more condition-specific models (e.g., models trained to generate predictions for the specific referral condition(s) that the patient had). In some embodiments, the referral prediction system may train one or more outcome-specific models (e.g., models trained to generate predictions for the specific outcome(s) experienced by the patient).

At block 1325, the referral prediction system determines whether there are any additional historic referrals that have not yet been used to train the model(s). If so, the method 1300 returns to block 1310 to select another record. Although the illustrated example depicts an iterative process (selecting and evaluating each historic referral in turn) for conceptual clarity, in some embodiments, the referral prediction system may train the models based on some or all of the referrals in parallel.

Returning to block 1325, if the referral prediction system determines that all available historic referrals have been used for training in the current iteration, the method 1300 continues to block 1330, where the referral prediction system determines whether one or more termination criteria are met. The termination criteria may generally include a wide variety of evaluations depending on the particular implementation. For example, in some embodiments, the referral prediction system may determine whether a defined number of iterations or epochs have been completed. In some embodiments, the referral prediction system determines whether a desired or preferred prediction accuracy has been reached. In some embodiments, the referral prediction system may determine whether a defined amount of resources have been spent training the model. In some embodiments, the referral prediction system determines whether any new referral records are ready for training.

If, at block 1330, the referral prediction system determines that the termination criteria are not met, the method 1300 returns to block 1305 to access a new (or the same) set of historic referral data. If the referral prediction system determines that the termination criteria are met, the method 1300 continues to block 1335, where the referral prediction system deploys the trained machine learning model(s) for inferencing.

As discussed above, deploying the model(s) may generally include any operations needed to prepare or provide the models for generating runtime inferences, such as transmitting the model parameter(s) to inferencing system(s), instantiating the model locally, and the like.

### Example Method for Using Machine Learning Models to Evaluate Referral Conditions

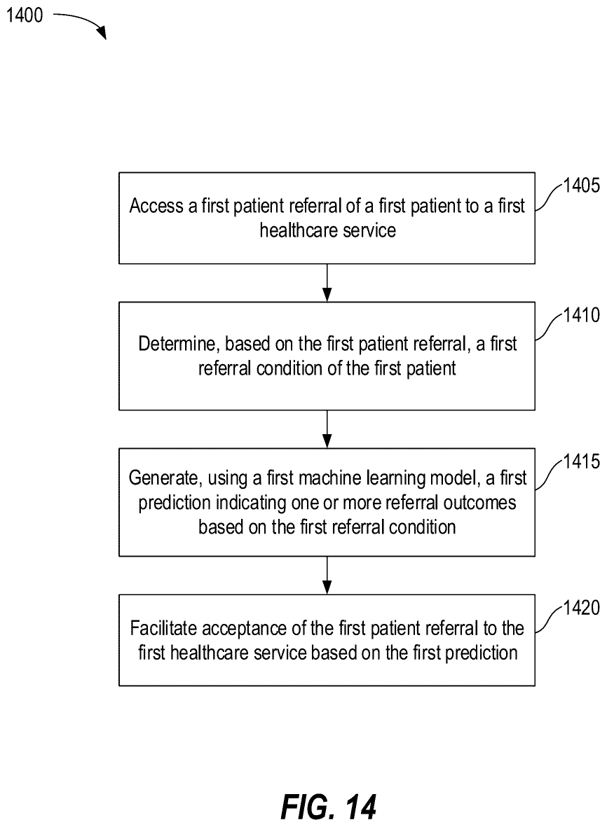

FIG. 14 is a flow diagram depicting an example method 1400 for using machine learning models to evaluate referral conditions, according to one embodiment of the present disclosure. In some embodiments, the method 1400 is performed by a referral prediction system, such as the referral prediction system 125 of FIG. 1.

At block 1405, a first patient referral of a first patient (e.g., patient 105 of FIG. 1) to a first healthcare service (e.g., service provider 120 of FIG. 1) is accessed.

At block 1410, a first referral condition (e.g., referral condition 210 of FIG. 2, referral condition 310 of FIG. 3, and/or referral condition 410 of FIG. 4) of the first patient is determined based on the first patient referral.

At block 1415, a first prediction (e.g., predicted outcomes 235 of FIG. 2, predicted outcomes 335 of FIG. 3, and/or predicted outcomes 435 of FIG. 4) indicating one or more referral outcomes is generated, using a first machine learning model (e.g., machine learning model 230 of FIGS. 2-4), based on the first referral condition.

At block 1420, acceptance of the first patient referral (e.g., referral response 130 of FIG. 1) to the first healthcare service is facilitated based on the first prediction.

### Example Method for Training Machine Learning Models

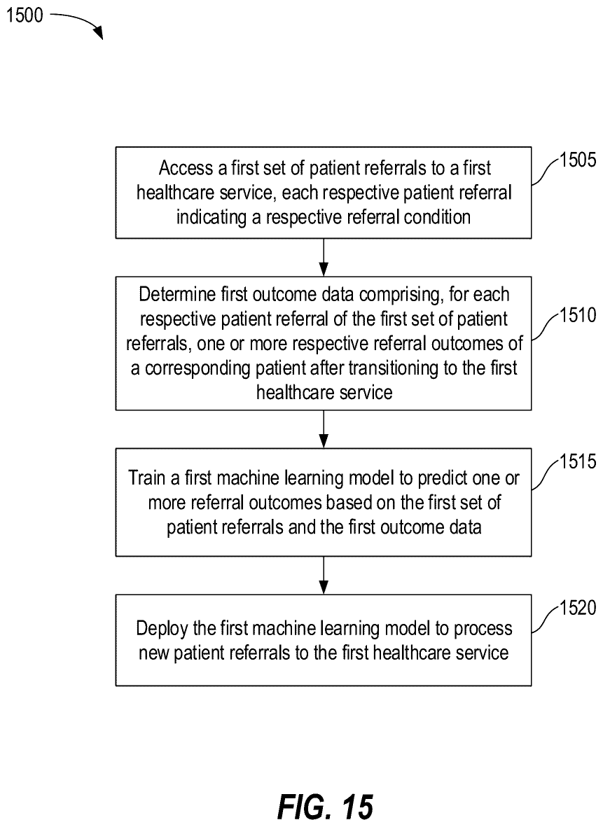

FIG. 15 is a flow diagram depicting an example method 1500 for training machine learning models, according to one embodiment of the present disclosure. In some embodiments, the method 1500 is performed by a referral prediction system, such as the referral prediction system 125 of FIG. 1.

At block 1505, a first set of patient referrals (e.g., historical referral data 510 of FIG. 5) to a first healthcare service (e.g., service provider 120 of FIG. 1) is accessed, each respective patient referral indicating a respective referral condition.

At block 1510, first outcome data (e.g., historical outcome data 513 of FIG. 5) is determined, comprising, for each respective patient referral of the first set of patient referrals, one or more respective referral outcomes of a corresponding patient after transitioning to the first healthcare service.

At block 1515, a first machine learning model (e.g., machine learning model 230 of FIG. 5) is trained to predict one or more referral outcomes based on the first set of patient referrals and the first outcome data.

At block 1520, the first machine learning model is deployed to process new patient referrals to the first healthcare service.

### Example Computing Device for Evaluating Referrals Using Machine Learning

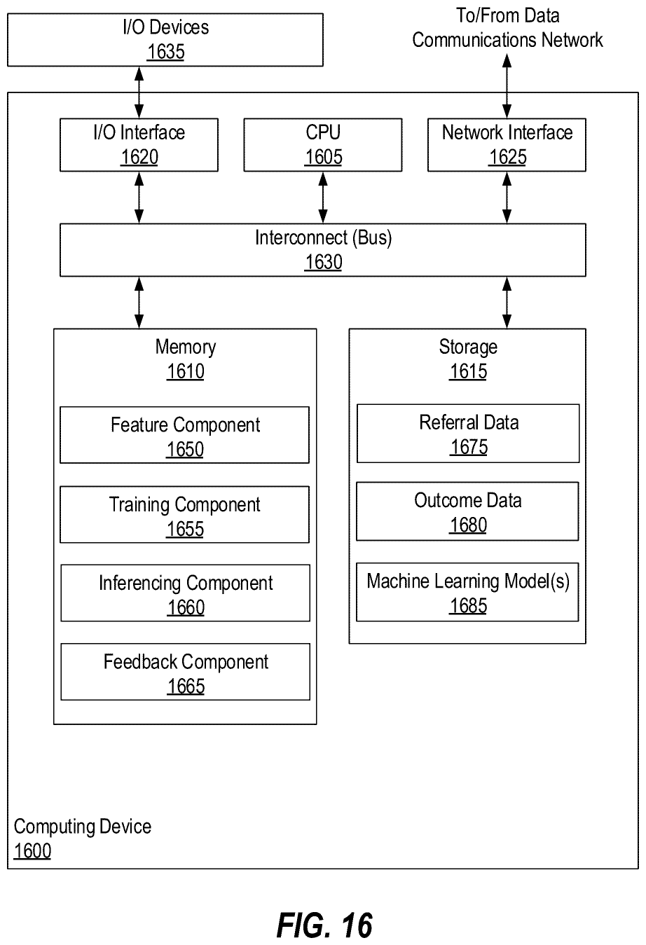

FIG. 16 depicts an example computing device 1600 configured to perform various aspects of the present disclosure. Although depicted as a physical device, in embodiments, the computing device 1600 may be implemented using virtual device(s), and/or across a number of devices (e.g., in a cloud environment). In some embodiments, the computing device 1600 corresponds to or implements a referral prediction system, such as the referral prediction system 125 of FIG. 1. In some embodiments, the computing device 1600 corresponds to or implements a feedback system, such as the feedback component 440 of FIG. 4. In some embodiments, the computing device 1600 corresponds to or implements a machine learning system, such as the machine learning system 515 of FIG. 5. Generally, the computing device 1600 may correspond to or implement any system that trains, updates, and/or uses machine learning models to predict patient outcomes based on healthcare referrals.

As illustrated, the computing device 1600 includes a CPU 1605, memory 1610, a network interface 1625, and one or more I/O interfaces 1620. Though not included in the depicted example, in some embodiments, the computing device 1600 also includes one or more storages. In the illustrated embodiment, the CPU 1605 retrieves and executes programming instructions stored in memory 1610, as well as stores and retrieves application data residing in memory 1610 and/or storage (not depicted). The CPU 1605 is generally representative of a single CPU and/or GPU, multiple CPUs and/or GPUs, a single CPU and/or GPU having multiple processing cores, and the like. The memory 1610 is generally included to be representative of a random access memory. In an embodiment, if storage is present, it may include any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1635 (such as keyboards, monitors, etc.) are connected via the I/O interface(s) 1620. Further, via the network interface 1625, the computing device 1600 can be communicatively coupled with one or more other devices and components (e.g., via a network, which may include the Internet, local network(s), and the like). As illustrated, the CPU 1605, memory 1610, network interface(s) 1625, and I/O interface(s) 1620 are communicatively coupled by one or more buses 1630.

In the illustrated embodiment, the memory 1610 includes a feature component 1650, a training component 1655, an inferencing component 1660, and a feedback component 1665, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1610, in embodiments, the operations of the depicted components (and others not illustrated) may be implemented using hardware, software, or a combination of hardware and software.

The feature component 1650 may generally be used to identify, extract, and/or generate feature data from patient data, as discussed above. For example, the feature component 1650 may evaluate patient data (e.g., patient data 110 of FIG. 1, patient data 205 of FIG. 2, patient data 305 of FIG. 3, and/or patient data 405 of FIG. 4) to generate model input (e.g., a feature tensor) that encodes this information for more efficient machine learning.

The training component 1655 may be used to train one or more machine learning models (such as machine learning models 1685) based on historic referral data, as discussed above. The inferencing component 1660 may be used to generate outcome predictions by processing patient referral data using one or more machine learning models (such as machine learning models 1685), as discussed above. The feedback component 1665 may be used to collect and/or generate feedback based on patient outcomes, allowing the feedback component 1665 and/or the training component 1655 to update the machine learning models continuously or periodically.

In the illustrated example, the storage 1615 includes referral data 1675, outcome data 1680, and machine learning models 1685. Although depicted as residing in storage 1614, the depicted data may be stored in any suitable location. In at least one embodiment, as discussed above, the referral data 1675, outcome data 1680, and machine learning models 1685 may be stored in separate repositories.

Generally, the referral data 1675 may include referral information for one or more historic referrals, as discussed above. For example, the referral data 1675 may correspond to training data, such as the training data 505 of FIG. 5, and may include information such as the historic referral data 510 of FIG. 5 and/or the historic outcome data 513 of FIG. 5. In some embodiments, the training component 1655 uses the referral data 1675 to train machine learning models 1685, as discussed above.

The outcome data 1680 (which may correspond to outcomes 445 of FIG. 4 and/or historical outcome data 513 of FIG. 5) may include information relating to outcomes experienced by patients during treatment by one or more healthcare services, such as whether they suffered an adverse event, whether they improved, and the like. The machine learning model(s) 1685 may generally correspond to one or more models trained to generate outcome predictions based on input patient data, such as the machine learning models 230 of FIGS. 2-5.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications (e.g., the components of the referral prediction system 125 of FIG. 1) or related data available in the cloud. For example, the referral prediction system 125 could execute on a computing system in the cloud and generate referral outcome predictions. In such a case, the referral prediction system 125 could store and execute machine learning models in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing a first patient referral of a first patient to a first healthcare service;
determining, based on the first patient referral, a first referral condition of the first patient;
generating, using a first machine learning model, a first prediction indicating one or more referral outcomes based on the first referral condition; and
facilitating acceptance of the first patient referral to the first healthcare service based on the first prediction.

2. The method of claim 1, wherein:
generating the first prediction comprises processing the first referral condition using the first machine learning model, and
the first prediction indicates, for each respective referral outcome of the one or more referral outcomes, a respective probability that the first patient will have the respective referral outcome if the first patient referral is accepted by the first healthcare service.

3. The method of claim 2, further comprising accessing a set of patient demographics for the first patient, wherein generating the first prediction is based further on processing the set of patient demographics using the first machine learning model.

4. The method of claim 1, wherein the one or more referral outcomes comprise at least one of:
(i) a prediction of whether the first patient will recover from the first referral condition,
(ii) a recovery timeline predicting a length of time until the first patient recovers from the first referral condition,
(iii) a prediction of whether the first patient will be hospitalized while being treated by the first healthcare service, or
(iv) a prediction of whether the first patient will become septic while being treated by the first healthcare service.

5. The method of claim 1, further comprising:
accessing a second patient referral to the first healthcare service;
determining, based on the second patient referral, a second referral condition of a second patient corresponding to the second patient referral;
generating, using the first machine learning model, a second prediction indicating one or more referral outcomes based on the second referral condition; and
facilitating declination of the second patient referral to the first healthcare service based on the second prediction.

6. The method of claim 5, further comprising:
generating, using a second machine learning model, a third prediction indicating one or more referral outcomes based on the second referral condition, wherein the second machine learning model was trained for a second healthcare service; and
indicating the second healthcare service in response to determining that the third prediction satisfies one or more criteria, as compared to the second prediction.

7. The method of claim 5, further comprising:
generating, using the first machine learning model, a third prediction indicating one or more referral outcomes based on the second referral condition, wherein the third prediction is generated by providing an indication of a second healthcare service as input to the first machine learning model; and
indicating the second healthcare service in response to determining that the third prediction satisfies one or more criteria, as compared to the second prediction.

8. The method of claim 5, further comprising:
determining one or more modifications to the first healthcare service that, if implemented, would improve predicted referral outcomes for the second referral condition; and
outputting an indication of the one or more modifications.

9. A method, comprising:
accessing a first set of patient referrals to a first healthcare service, each respective patient referral indicating a respective referral condition;
determining first outcome data comprising, for each respective patient referral of the first set of patient referrals, one or more respective referral outcomes of a corresponding patient after transitioning to the first healthcare service;
training a first machine learning model to predict one or more referral outcomes based on the first set of patient referrals and the first outcome data; and
deploying the first machine learning model to process new patient referrals to the first healthcare service.

10. The method of claim 9, wherein training the first machine learning model comprises, for a first patient referral of the first set of patient referrals:
generating a predicted referral outcome based on processing a first referral condition of the first patient referral using the first machine learning model;
determining a difference between the predicted referral outcome and a first referral outcome of the first patient referral; and
updating one or more parameters of the first machine learning model based on the difference.

11. The method of claim 10, further comprising accessing a set of patient demographics for a first patient indicated by the first patient referral, wherein generating the predicted referral outcome is based further on processing the set of patient demographics using the first machine learning model.

12. The method of claim 9, wherein the first outcome data comprises, for each respective patient referral, at least one of:
(i) a respective indication of whether the corresponding patient recovered from a respective referral condition,
(ii) a respective recovery timeline indicating a length of time until the corresponding patient recovered from a respective referral condition, or
(iii) a respective indication of whether the corresponding patient was hospitalized while being treated by the first healthcare service.

13. The method of claim 9, wherein training the first machine learning model to predict one or more referral outcomes comprises training the first machine learning model to predict a plurality of referral outcomes.

14. The method of claim 9, further comprising:
accessing a second set of patient referrals to a second healthcare service;
determining second outcome data for each respective patient referral of the second set of patient referrals; and
training a second machine learning model to predict one or more referral outcomes based on the second set of patient referrals and the second outcome data.

15. The method of claim 9, further comprising:
accessing a second set of patient referrals to a second healthcare service;
determining second outcome data for each respective patient referral of the second set of patient referrals; and
training the first machine learning model to predict one or more referral outcomes based on the second set of patient referrals and the second outcome data while using an indication of the second healthcare service as input to the first machine learning model.

16. One or more non-transitory computer-readable media comprising computer-executable instructions that, when executed by one or more processors of one or more processing systems, cause the one or more processing systems to perform an operation comprising:
accessing a first patient referral of a first patient to a first healthcare service;
determining, based on the first patient referral, a first referral condition of the first patient;
generating, using a first machine learning model, a first prediction indicating one or more referral outcomes based on the first referral condition; and
facilitating acceptance of the first patient referral to the first healthcare service based on the first prediction.

17. The non-transitory computer-readable media of claim 16, wherein:
generating the first prediction comprises processing the first referral condition using the first machine learning model, and
the first prediction indicates, for each respective referral outcome of the one or more referral outcomes, a respective probability that the first patient will have the respective referral outcome if the first patient referral is accepted by the first healthcare service.

18. The non-transitory computer-readable media of claim 16, the operation further comprising:
accessing a second patient referral to the first healthcare service;
determining, based on the second patient referral, a second referral condition of a second patient corresponding to the second patient referral;
generating, using the first machine learning model, a second prediction indicating one or more referral outcomes based on the second referral condition; and
facilitating declination of the second patient referral to the first healthcare service based on the second prediction.

19. The non-transitory computer-readable media of claim 18, the operation further comprising:
generating a third prediction indicating one or more referral outcomes based on the second referral condition, wherein the third prediction corresponds to a second healthcare service; and
indicating the second healthcare service in response to determining that the third prediction satisfies one or more criteria, as compared to the second prediction.

20. The non-transitory computer-readable media of claim 18, the operation further comprising:
determining one or more modifications to the first healthcare service that, if implemented, would improve predicted referral outcomes for the second referral condition; and
outputting an indication of the one or more modifications.
